(19) United States
(12) Patent Application Publication
(10) Pub. No.: US 2006/0177480 A1
(43) Pub. Date: Aug. 10, 2006

(54) Drug-eluting biodegradable stent

(76) Inventors: Hsing-Wen Sung, HsinChu (TW); Mei-Chin Chen, Taipei County (TW); Hosheng Tu, Newport Beach, CA (US)

Correspondence Address: HOSHENG TU, 15 RIEZ, NEWPORT BEACH, CA, 92657-0116, US

(21) Appl. No.: 11/130,787

(22) Filed: May 17, 2005

Related U.S. Application Data
(63) Continuation-in-part of application No. 10/906,239, filed on Feb. 10, 2005.

(51) Int. Cl. A61F 2/00

(52) U.S. Cl. 424/426

(57) ABSTRACT

The present invention relates to a biodegradable stent comprising a luminal surface portion with a second degree of crosslink, an outer surface portion with a first degree of crosslink, and a wall between the luminal and outer surface portions, wherein the wall comprises a crosslinked material characterized by the first degree of crosslink not less than the second degree of crosslink.

### CROSS-REFERENCE TO RELATED APPLICATIONS

This application is a continuation-in-part application of U.S. patent application Ser. No. 10/906,239, filed Feb. 10, 2005, the entire contents of which are incorporated herein by reference.

### FIELD OF THE INVENTION

The present invention generally relates to crosslinkable collagen, chitosan, gelatin, and/or fibrin glue loaded with bioactive agents configured suitably for therapeutic purposes, and more particularly, the present invention relates to a drug-loaded biodegradable stent and delivery means for treating atherosclerotic or vulnerable plaques of a patient.

### BACKGROUND OF THE INVENTION

Crosslinking of biological molecules is often desired for optimal effectiveness in biomedical applications. For example, collagen, which constitutes the structural framework of biological tissue, has been extensively used for manufacturing bioprostheses and other implanted structures, such as vascular grafts, wherein it provides a good medium for cell infiltration and proliferation. However, biomaterials derived from collagenous tissue must be chemically modified and subsequently sterilized before they can be implanted in humans. The fixation, or crosslinking, of collagenous tissue increases strength and reduces antigenicity and immunogenicity. In one aspect of the present invention, crosslinking of a drug-containing biological material with genipin enables the resulting material with less antigenicity or immunogenicity, wherein the biological material comprises collagen, gelatin, elastin or tropoelastin, chitosan, N, O, carboxylmethyl chitosan (NOCC), and the like (such as fibrin glue, biological sealant, fibronectin derivatives and combination thereof) that has at least one amino functional group for reaction with genipin.

Collagen sheets are also used as wound dressings, providing the advantages of high permeability to water vapor and rapid wound healing. Disadvantages include low tensile strength and easy degradation of collagen by collagenase. Crosslinking of collagen sheets reduces cleavage by collagenase and improves tensile strength. In one aspect, a collagen strip derived of crosslinked drug-containing collagen sheets may be used to load on the periphery of a stent or be reinforced as a drug-eluting stent to mitigate restenosis or other abnormality. In a further aspect, the collagen sheet or collagen strip may be made of solidifiable collagen. Same is feasible with other biological material of the present disclosure.

Clinically, biological tissue has been used in manufacturing heart valve prostheses, small-diameter vascular grafts, ligament replacements, and biological patches, among others. However, the biological tissue has to be fixed with a crosslinking or chemically modifying agent and subsequently sterilized before they can be implanted in humans. The fixation of biological tissue or collagen is to reduce antigenicity and immunogenicity and prevent enzymatic degradation. Various crosslinking agents have been used in fixing biological tissue. These crosslinking agents are mostly synthetic chemicals such as formaldehyde, glutaraldehyde, dialdehyde starch, glyceraldehydes, cyanamide, diimides, diisocyanates, dimethyl adipimidate, carbodiimide, and epoxy compound. However, these chemicals are all highly cytotoxic which may impair the biocompatibility of biological tissue. Of these, glutaraldehyde is known to have allergenic properties, causing occupational dermatitis and is cytotoxic at concentrations greater than 10-25 ppm and as low as 3 ppm in tissue culture. It is therefore desirable to provide a crosslinking agent (that is, a crosslinking reagent) suitable for use in biomedical applications that is within acceptable cytotoxicity and that forms stable and biocompatible crosslinked products.

An example of a genipin-crosslinked heart valve is reported by Sung et al., a co-inventor of the present invention, (Journal of Thoracic and Cardiovascular Surgery 2001; 122:1208-1218) entitled Reconstruction of the right ventricular outflow tract with a bovine jugular vein graft fixed with a naturally occurring crosslinking agent (genipin) in a canine model, the entire contents of which are incorporated herein by reference. Sung et al. herein discloses genipin and its crosslinking ability to a collagen-containing biological tissue heart valve used in an animal implantation study, which demonstrates valve's safety and efficacy with genipin treatment.

To achieve this goal, a naturally occurring crosslinking agent (genipin) has been used to fix biological tissue. The cytotoxicity of genipin was previously studied in vitro using 3T3 fibroblasts, indicating that genipin is substantially less cytotoxic than glutaraldehyde (Sung H W et al., J Biomater Sci Polymer Edn 1999; 10:63-78). Additionally, the genotoxicity of genipin was tested in vitro using Chinese hamster ovary (CHO—K1) cells, suggesting that genipin does not cause clastogenic response in CHO—K1 cells (Tsai CC et al., J Biomed Mater Res 2000; 52:58-65), incorporated herein by reference. A biological material (including collagen-containing or chitosan-containing substrate) treated with genipin resulting in acceptable cytotoxicity is a first requirement to biomedical applications.

U.S. Pat. No. 6,545,042 by one of the present inventors, the entire contents of which are incorporated herein by reference, discloses an acellular tissue providing a natural microenvironment for host cell migration, in vitro endothelialization, or in vivo endothelialization to promote and accelerate tissue regeneration. The genipin-treated biological biomaterial has reduced antigenicity and immunogenicity.

Atherosclerosis causes a partial blockage of the blood vessels that supply the heart with nutrients. Atherosclerotic blockage of blood vessels often leads to hypertension, ischemic injury, stroke, or myocardial infarction. Typically angioplasty and/or stenting is a remedy for such a disease, however, restenosis does occur in 30-40 percent patients resulting from intimal smooth muscle cell hyperplasia. The underlying cause of the intimal smooth muscle cell hyperplasia is mainly vascular smooth muscle injury and disruption of the endothelial lining.

Vascular injury causing intimal thickening can be from mechanical injuries due to angioplasty and/or stenting. Intimal thickening following balloon catheter injury has been studied in animals as a model for arterial restenosis that occurs in human patients following balloon angioplasty. Injury is followed by a proliferation of the medial smooth muscle cells, after which many of them migrate into the intima through fenestration in the internal elastic lamina and proliferation to form a neointimal lesion.

Vascular stenosis can be detected and evaluated using angiographic or sonographic imaging techniques and is often treated by percutaneous transluminal coronary angioplasty (balloon catheterization). Within a few months following angioplasty, however, the blood flow is reduced in approximately 30-40 percent of these patients as a result of restenosis caused by a response to mechanical vascular injury suffered during the angioplasty or stenting procedure, as described above.

In an attempt to prevent restenosis or reduce intimal smooth muscle cell proliferation following angioplasty, numerous pharmaceutical agents have been employed clinically, concurrent with or following angioplasty. Most pharmaceutical agents employed in an attempt to prevent or reduce the extent of restenosis have been unsuccessful. The following list identifies several of the agents for which favorable clinical results have been reported: lovastatin; thromboxane A₂ synthetase inhibitors such as DP-1904; eicosapentanoic acid; ciprostene (a prostacyclin analog); trapidil (a platelet derived growth factor)]; angiotensin convening enzyme inhibitors; and low molecular weight heparin, the entire contents of the above-referred drugs and their therapeutic effects are incorporated herein by reference. It is one aspect of the present invention to provide site-specific administration of the pharmaceutical agents disclosed in this invention to the injury site for effective therapy via a genipin-crosslinked collagen-containing or chitosan-containing stent or implant, which can be biodegradable.

Many compounds have been evaluated in a standard animal model. The immunosuppressive agent cyclosporin A has been evaluated and has produced conflicting results. Jonasson reported that cyclosporin A caused an inhibition of the intimal proliferative lesion following arterial balloon catheterization in vivo, but did not inhibit smooth muscle cell proliferation in vitro. It was reported that when de-endothelialized rabbits were treated with cyclosporin A, no significant reduction of intimal proliferation was observed in vivo. Additionally, intimal accumulations of foamy macrophages, together with a number of vacuolated smooth muscle cells in the region adjacent to the internal elastic lamina were observed, indicating that cyclosporin A may modify and enhance lesions that form at the sites of arterial injury.

Morris et al. in U.S. Pat. No. 5,516,781 disclosed Rapamycin (also known as sirolimus), a macrocyclic triene antibiotic produced by Streptomyces hygroscopicus that has been shown to prevent the formation of humoral (IgE-like) antibodies in response to an albumin allergic challenge, inhibit murine T-cell activation, prolong survival time of organ gratis in histoincompatible rodents, and inhibit transplantation rejection in mammals. Rapamycin blocks calcium-dependent, calcium-independent, cytokine-independent and constitutive T and B cell division at the G1-S interface. Rapamycin inhibits gamma-interferon production induced by II-1 and also inhibits the gamma-interferon induced expression of membrane antigen. Arterial thickening following transplantation, known as CGA, is a limiting factor in graft survival that is caused by a chronic immunological response to the transplanted blood vessels by the transplant recipient's immune system.

Further, Morris et al. in U.S. Pat. No. 5,516,781 claims the invention is distinct from the use of rapamycin for preventing CGA, in that CGA does not involve injury to the recipients' own blood vessels; it is a rejection type response. The '781 patent is related to vascular injury to native blood vessels. The resulting intimal smooth muscle cell proliferation does not involve the immune system, but is growth factor mediated. For example, arterial intimal thickening after balloon catheter injury is believed to be caused by growth factor (PGDF, bFGF, TGFb, IL-1 and others)-induced smooth muscle cell proliferation and migration. The above-cited U.S. Pat. No. 5,516,781 is incorporated herein by reference.

In the past, polymer or plastic materials have been used as a carrier for depositing a drug or pharmaceutical agent onto the periphery of a stent to treat restenosis. One example is U.S. Pat. No. 6,544,544 to Hunter et al., the entire contents of which are incorporated herein by reference. Hunter et al. discloses a method for treating a tumor excision site, comprising administering to a patient a composition comprising paclitaxel, or an analogue or derivative thereof, to the resection margin of a tumor subsequent to excision, such that the local recurrence of cancer and the formation of new blood vessels at the site is inhibited. The composition further comprises a polymer, wherein the polymer may comprise poly(caprolactone), poly(lactic acid), poly(ethylene-vinyl acetate), and poly(lactic-co-glycolic)acid.

In another example, Biocompatibles PC (phosphorylcholine by Biocompatibles, London, England) has been added as a drug carrier or surface modifier for treating tissue injury due to angioplasty and/or stenting. The technique comprises a hydrophobic component that aids in the initial adhesion and film-formation of the polymer onto the stainless steel stent substrate, and other groups allow cross-linking both within the polymer and with the stent surface to achieve firm anchorage. The coating is thus tenaciously adhered to the stent and can survive balloon expansion without damage. A therapeutic drug can be loaded within the coated substrate, such as phosphorylcholine. Some aspects of the invention relate to a biodegradable stent made of a biological material selected from a group consisting of chitosan, collagen, elastin, gelatin, fibrin glue, biological sealant, and combination thereof, wherein the biological material is further mixed with phosphorylcholine as raw material for the biodegradable stent.

Drugs are usually loaded, admixed or entrapped physically within the polymer framework for slow drug release. The plastic polymer which is suitable as a drug carrier may not be biocompatible, whereas some biocompatible plastic polymer may not be able to contain a specific drug and release drug in an effective timely amount for effective therapy. Therefore, there is a clinical need to have a biocompatible drug carrier that releases an effective quantity of drug over a period of time for prolonged therapeutic effects, such as a biodegradable biological material.

U.S. Pat. No. 5,085,629 issued on Feb. 4, 1992, the entire contents of which are incorporated herein by reference, discloses a biodegradable, biocompatible, resorbable infusion stent comprising a terpolymer of: (a) L(−)lactide, (b) glycolide, and (c) epsilon-caprolactone. This invention includes a method for treating ureter obstructions or impairments by utilizing a biodegradable, biocompatible, resorbable infusion stent, and a method for controlling the speed of resorption of the stent. A ureter stent that is made of a biodegradable and biocompatible material would assure its safe and innocuous disappearance without the need for a second surgical procedure for its removal after it has completed its function.

It is generally agreed that an ideal stent for vulnerable plaque treatment should: be biodegradable after serving its purpose and its breakdown products must be biocompatible; possess physical properties sufficient to perform its mechanical function; have sufficient longitudinal flexibility to facilitate insertion; and be able to deliver drugs locally to prevent restenosis or treat plaque.

U.S. Pat. No. 5,464,450 issued on Nov. 7, 1995, the entire contents of which are incorporated herein by reference, discloses a stent made of biodegradable material including a drug that is released at a rate controlled by the rate of degradation of the biodegradable material. The stent includes a main body of a generally tubular shape. The main body may further include a plurality of apertures extending therethrough and a slot defined by opposing edges which permits insertion and positioning of the stent.

U.S. Pat. No. 6,200,335 issued on Mar. 13, 2001 and U.S. Pat. No. 6,632,242 issued on Oct. 14, 2003, the entire contents of which are incorporated herein by reference, disclose a stent inserted into the vessel of a living body including a tubular member constituting a passageway from one end to its opposite end. The tubular member includes a main mid portion and low tenacity portions formed integrally with both ends of the main mid portion. The low tenacity portions are lower in tenacity than the main mid portion. These low tenacity portions are formed so as to have the Young's modulus approximate to that of the vessel of the living body in which is inserted the stent, so that, when the stent is inserted into the vessel, it is possible to prevent stress concentrated portions from being produced in the vessel.

In accordance with the present invention there is provided chemically treated collagen-containing or chitosan-containing biological implant or stent loaded with at least one bioactive agent which have shown to exhibit many of the desired characteristics important for optimal therapeutic function. In particular, the crosslinked collagen/chitosan-drug compound with drug slow release capability may be suitable in treating atherosclerosis, vulnerable plaques, and other therapeutic applications.

### SUMMARY OF THE INVENTION

In general, it is an object of the present invention to provide a biological substance configured and adapted for drug slow release. In one aspect of the present invention, the biological substance may be a cardiovascular stent or implant. The “biological substance” is herein intended to mean a substance made of drug-containing biological material that is, in one preferred embodiment, solidifiable or hardenable upon change of environmental condition(s) and is biocompatible post-crosslinking with a crosslinker, such as genipin, its derivatives, analog (for example, aglycon geniposidic acid), stereoisomers and mixtures thereof. In one embodiment, the crosslinker may further comprise epoxy compounds, dialdehyde starch, glutaraldehyde, formaldehyde, dimethyl suberimidate, carbodiimides, succinimidyls, diisocyanates, acyl azide, reuterin, ultraviolet irradiation, dehydrothermal treatment, tris(hydroxymethyl)phosphine, ascorbate-copper, glucose-lysine and photo-oxidizers, and the like. The “biological material” is intended herein to mean collagen, gelatin, elastin or tropoelastin, chitosan, NOCC (N, O, carboxylmethyl chitosan), fibrin glue, biological sealant, and the like that could be crosslinked with a crosslinker (also known as a crosslinking agent).

In one embodiment, the process of preparing a biological substance comprises steps, in combination, of loading drugs with the biological material, shaping the drug-containing biological material, followed by crosslinking with genipin. The genipin referred herein is broadly consisted of the naturally occurring compound as shown in FIG. 1 and its derivatives, analog, stereoisomers and mixtures thereof. In another embodiment, the drug-containing biological material is further coated, adhered or loaded onto a physical construct or apparatus before or after crosslinking with a crosslinker (such as genipin). The biological material may be in a form or phase of solution, paste, gel, suspension, colloid or plasma that may be solidified or hardened thereafter.

It is another object of the present invention to provide a method for drug slow release from a medical device comprising entrapping drug within a biological material crosslinked with genipin or other crosslinkers. The medical device can be a stent (biodegradable or non biodegradable), a non-stent implant or prosthesis, or a percutaneous device such as a catheter, a wire, a cannula, an endoscopic instrument or the like for the intended drug slow release. In one embodiment, the non-stent implant may comprise biological implant, non-biological implant, annuloplasty rings, heart valve prostheses, venous valve bioprostheses, orthopedic implants, dental implants, ophthalmology implants, cardiovascular implants, and cerebral implants.

It is a further object of the present invention to provide a method for drug slow release from an implant comprising chemically bonding (ionically or covalently) drug within a biological material crosslinked with genipin, wherein the drug has an amine or amino group branch. In one aspect of the present invention, the amine or amino group of the drug is reacted with the amino group of a suitable biological material through a crosslinker.

Some aspects of the invention relate to a vascular stent, comprising a biodegradable or non biodegradable stent base coated with at least one layer of partially crosslinked collagen. In one embodiment, the at least one collagen layer comprises a drug or drugs, each collagen layer comprising different drug content, drug type, drug concentration, or combination thereof. In another embodiment, the stent base is made of biological material. Some preferred aspects of the invention relate to a medical device comprising a biodegradable apparatus having a surface, at least one bioactive agent, and biological material loaded onto at least a portion of the surface of the apparatus, the biological material comprising the at least one bioactive agent, wherein the biological material is crosslinked with a crosslinking agent or with ultraviolet irradiation.

Some aspects of the invention relate to a method of treating a target tissue of a patient comprising: providing a biodegradable stent made of at least one layer or zone of biological material, the biological material comprising at least one bioactive agent; crosslinking the biological material; and delivering the stent to the target tissue; and releasing the bioactive agent for treating the target tissue. In a further embodiment, the stent comprises a first layer or zone of a first biological material with a first bioactive agent and a second layer or zone of a second biological material with a second bioactive agent.

Some aspects of the invention relate to a biodegradable stent for treating vulnerable plaques of a patient comprising a plurality of layers or zones, each layer or zone comprising its own specific biodegradation rate and its specific drug loading characteristics, wherein the drug loading characteristics is meant to include drug type, drug amount, drug releasing rate, combination of more than one drug, and the like. In one embodiment, the layers and zones are configured and arranged, in combination, radially, circumferentially and longitudinally. In one embodiment, the layer is conveniently defined herein in a radial manner whereas the zone is defined herein in a circumferential or longitudinal manner. In other words, in the radial direction, there may be one or more layers whereas in the circumferential or longitudinal direction, there may be one or more zones.

Some aspects of the invention relate to a drug-loaded biodegradable stent, wherein the biodegradation rate of a first layer or zone is equal to or faster than the biodegradation rate of a second layer or zone.

Some aspects of the invention relate to a biodegradable stent of the invention, wherein at least one of the first and the second layer or zone is made of a shape memory polymer or biodegradable shape memory polymer.

Some aspects of the invention relate to a biodegradable stent of the invention, wherein at least one of the first and the second layer or zone further comprises a biological material, wherein the biological material is phosphorylcholine.

Some aspects of the invention relate to a biodegradable stent of the invention, wherein the biodegradable material is made of a material selected from a group consisting of polylactic acid (PLA), polyglycolic acid (PGA), poly(D,L-lactide-co-glycolide), polycaprolactone, and co-polymers thereof.

Some aspects of the invention relate to a biodegradable stent of the invention, wherein the biodegradable material is made of a material selected from a group consisting of polyhydroxy acids, polyalkanoates, polyanhydrides, polyphosphazenes, polyetheresters, polyesteramides, polyesters, and polyorthoesters.

Some aspects of the invention relate to a biodegradable stent of the invention, wherein at least one of the first and the second layer or zone comprises at least one bioactive agent.

Some aspects of the invention relate to a biodegradable stent of the invention, wherein at least one of the first and the second layer or zone comprises a plurality of bioactive agents.

Some aspects of the invention relate to a biodegradable stent of the invention, wherein at least one of the first and the second layer or zone comprises a plurality of bioactive agents in distinct multi-layers.

Some aspects of the invention relate to a biodegradable stent of the invention, wherein phosphorylcholine is coated at the outermost layer of the stent.

Some aspects of the invention relate to a biodegradable stent of the invention, wherein at least one of the first and the second layer or zone comprises at least one bioactive agent, wherein the at least one bioactive agent is selected from a group consisting of analgesics/antipyretics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, antimicrobials, anti-infectives, and combination thereof.

Some aspects of the invention relate to a biodegradable stent of the invention, wherein at least one of the first and the second layer or zone comprises at least one bioactive agent, wherein the at least one bioactive agent is selected from a group consisting of actinomycin D, paclitaxel, vincristin, methotrexate, and angiopeptin, batimastat, halofuginone, sirolimus, tacrolimus, everolimus, ABT-578, tranilast, dexamethasone, and mycophenolic acid.

Some aspects of the invention relate to a biodegradable stent of the invention, wherein at least one of the first and the second layer or zone comprises at least one bioactive agent, wherein the at least one bioactive agent is selected from a group consisting of lovastatin, thromboxane A₂ synthetase inhibitors, eicosapentanoic acid, ciprostene, trapidil, angiotensin convening enzyme inhibitors, aspirin, and heparin.

Some aspects of the invention relate to a biodegradable stent of the invention, wherein at least one of the first and the second layer or zone comprises at least one bioactive agent, wherein the at least one bioactive agent is selected from a group consisting of allicin, ginseng extract, ginsenoside Rg1, flavone, ginkgo biloba extract, glycyrrhetinic acid, and proanthocyanides.

Some aspects of the invention relate to a biodegradable stent of the invention, wherein at least one of the first and the second layer or zone comprises at least one bioactive agent, wherein the at least one bioactive agent comprises ApoA-I Milano or recombinant ApoA-I Milano/phospholipid complexes.

Some aspects of the invention relate to a biodegradable stent of the invention, wherein at least one of the first and the second layer or zone comprises at least one bioactive agent, wherein the at least one bioactive agent comprises biological cells or endothelial progenitor cells.

Some aspects of the invention relate to a biodegradable stent of the invention, wherein at least one of the first and the second layer or zone comprises at least one bioactive agent, wherein the at least one bioactive agent comprises lipostabil.

Some aspects of the invention relate to a biodegradable stent of the invention, wherein at least one of the first and the second layer or zone comprises at least one bioactive agent, wherein the at least one bioactive agent comprises a growth factor, wherein the growth factor is selected from a group consisting of vascular endothelial growth factor, transforming growth factor-beta, insulin-like growth factor, platelet derived growth factor, fibroblast growth factor, and combination thereof.

Some aspects of the invention relate to a biodegradable stent made of a biological material selected from a group consisting of chitosan, collagen, elastin or tropoelastin, gelatin, fibrin glue, and combination thereof. In a further embodiment, the stent has a collapse pressure of at least 1 bar. In a further embodiment, the stent is further crosslinked with a crosslinking agent or with ultraviolet or gamma irradiation. In a further embodiment, the biological material is crosslinked with a crosslinking agent, wherein the crosslinking agent is genipin, its analog, derivatives, and combination thereof. In a further embodiment, the biological material is crosslinked with a crosslinking agent, wherein the crosslinking agent is selected from a group consisting of formaldehyde, glutaraldehyde, dialdehyde starch, glyceraldehydes, cyanamide, diimides, diisocyanates, dimethyl adipimidate, carbodiimide, epoxy compound, reuterin, and mixture thereof.

Some aspects of the invention relate to a biodegradable stent made of a biological material selected from a group consisting of chitosan, collagen, elastin or tropoelastin, gelatin, fibrin glue, biological sealant, and combination thereof, wherein the biological material is further mixed with a substrate as raw material for the biodegradable stent, the substrate being selected from a group consisting of poly(L-lactic acid), polyglycolic acid, poly(D,L-lactide-co-glycolide), polycaprolactone, mixture thereof, and co-polymers thereof. In a further embodiment, the biological material is further mixed with a substrate as raw material for the biodegradable stent, the substrate is phosphorylcholine. In a further embodiment, the stent is a spiral stent or a double spiral stent.

Some aspects of the invention relate to a biodegradable stent made of a biological material selected from a group consisting of chitosan, collagen, elastin or tropoelastin, gelatin, fibrin glue, biological sealant, and combination thereof, wherein the biological material is further mixed with at least one bioactive agent. In a further embodiment, the at least one bioactive agent is selected from a group consisting of analgesics/antipyretics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, antimicrobials, anti-infectives, angiogenesis factors, and anti-angiogenesis factors. In a further embodiment, the at least one bioactive agent is selected from a group consisting of actinomycin D, paclitaxel, vincristin, methotrexate, and angiopeptin, batimastat, halofuginone, sirolimus, tacrolimus, everolimus, ABT-578, tranilast, dexamethasone, and mycophenolic acid. In a further embodiment, the at least one bioactive agent is selected from a group consisting of lovastatin, thromboxane A₂ synthetase inhibitors, eicosapentanoic acid, ciprostene, trapidil, angiotensin convening enzyme inhibitors, aspirin, and heparin. In a further embodiment, the at least one bioactive agent is selected from a group consisting of allicin, ginseng extract, ginsenoside Rg1, flavone, ginkgo biloba extract, glycyrrhetinic acid, and proanthocyanides. In a further embodiment, the at least one bioactive agent comprises ApoA-I Milano or recombinant ApoA-I Milano/phospholipid complexes. In a further embodiment, the at least one bioactive agent comprises biological cells or endothelial progenitor cells. In a further embodiment, the at least one bioactive agent comprises lipostabil. In a further embodiment, the at least one bioactive agent comprises a growth factor, wherein the growth factor is selected from a group consisting of vascular endothelial growth factor, transforming growth factor-beta, insulin-like growth factor, platelet derived growth factor, fibroblast growth factor, and combination thereof.

Some aspects of the invention relate to a biodegradable stent made of a biological material selected from a group consisting of chitosan, collagen, elastin or tropoelastin, gelatin, fibrin glue, biological sealant, and combination thereof, wherein the stent comprises a plurality of layers made of the biological material. In one embodiment, the plural layers are distinct layers. In another embodiment, the plural layers are non-distinct layers.

Some aspects of the invention relate to a biodegradable stent made of a biological material selected from a group consisting of chitosan, collagen, elastin or tropoelastin, gelatin, fibrin glue, biological sealant, and combination thereof, wherein the stent comprises a plurality of layers, each layer is made of the biological material with at least one bioactive agent. In one embodiment, a first of the plural layers is made of the biological material composition different from that of a second layer.

Some aspects of the invention relate to a method for treating vulnerable plaques of a patient, comprising: providing a biodegradable stent made of a biological material selected from a group consisting of chitosan, collagen, elastin or tropoelastin, gelatin, fibrin glue, biological sealant, and combination thereof; deploying the biodegradable stent to the vulnerable plaques site; and releasing the at least one bioactive agent for treating the vulnerable plaques. In an alternate embodiment, the method further comprises a step of crosslinking the biodegradable stent with a crosslinking agent or with ultraviolet or gamma irradiation.

Some aspects of the invention relate to a biodegradable stent in a non-tubular cylindrical shape that has a first diameter or circumference length before contacting water and a second diameter or circumference length after contacting water, wherein the second diameter or circumference length is at least 5% more than the first diameter or circumference length.

Some aspects of the invention relate to a crosslinked biodegradable stent or implant comprising at least one layer or zone of biological material, the biological material comprising at least one bioactive agent and being crosslinked with means for crosslinking (permanently or reversibly) the biological material. In one embodiment, the crosslinked biodegradable stent is a cylindrical or non-tubular construct comprising a plurality of open-ring stent members configured in a cylindrical manner.

Some aspects of the present invention relate to a method of treating a target tissue of a patient comprising: providing a biodegradable stent made of biological material, wherein the stent is not in a tubular shape before implantation, the biological material comprising at least one bioactive agent; crosslinking the biological material; and delivering the stent to the target tissue and releasing the bioactive agent for treating the target tissue. In a further embodiment, the biological material is selected from a group consisting of collagen, gelatin, elastin or tropoelastin, chitosan, NOCC, low MW chitosan, fibrin glue, biological sealant, chitosan-alginate complex, chitosan-glycerol complex, and combination thereof.

Some aspects of the present invention relate to a crosslinked biodegradable stent comprising at least one bioactive agent, wherein the stent is a non-tubular shape in a first position before implantation and a tubular shape in a second position after implantation. In a further embodiment, at least a portion of the stent is made of biological material that is selected from a group consisting of collagen, gelatin, elastin or tropoelastin, chitosan, NOCC, low MW chitosan, fibrin glue, biological sealant, chitosan-alginate complex, chitosan-glycerol complex, and combination thereof. In still a further embodiment, the biological material is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof. In one further embodiment, the biological material is crosslinked with a means for crosslinking the material, the means comprising exposing the material to ultraviolet irradiation, dehydrothermal treatment, tris(hydroxymethyl)phosphine, ascorbate-copper, glucose-lysine or photo-oxidizers. In a preferred embodiment, the biological material is crosslinked with a reversible crosslinking agent selected from a group consisting of polyphenolic compounds, proanthocyanidin, epigallocatechin gallate, epicatechin, epigallocatechin, epicatechin gallate, and combination thereof.

Some aspects of the present invention relate to a stent comprising at least one bioactive agent, wherein the stent is not in a tubular shape before implantation. Some aspects of the present invention relate to a biodegradable stent comprising at least one bioactive agent, wherein the biodegradable stent is in a sheet or slightly curved sheet shape before implantation and is in a tubular shape after implantation. In one embodiment, the sheet comprises a plurality of holes larger than about 100 microns, preferably larger than 500 microns.

Some aspects of the invention relate to a biodegradable vascular stent for treating atherosclerosis comprising a composite material, wherein the composite material comprises a non-metallic base material and a plurality of reinforcing elements sufficient for enhancing the radial strength (also known as hoop strength after implantation) of the stent. In a further embodiment, the base material is a crosslinked material. In one embodiment, the base material forms an essentially continuous phase while the reinforcing elements form a dispersed discrete phase within the continuous phase.

In one embodiment, the reinforcing elements of the reinforced biodegradable stent are characterized in that a first Young's modulus of at least a portion of the elements is greater than a second Young's modulus of the base material. In one further embodiment, the reinforcing elements are sized and configured with a longitudinal length and a traverse length, the reinforcing elements being selected from a group consisting of fibers, strips, filaments, elongate meshes, and combination thereof, wherein the longitudinal length of the elements is in the range of 0.05 μm to 250 μm and the traverse length of the elements is in the range of 0.1 μm to 500 μm.

In a further embodiment, the reinforcing elements of the reinforced biodegradable stent are made of a biodegradable material selected from a group consisting of biodegradable metals, biodegradable metal alloys, biodegradable metal oxides, biodegradable polymers, and biodegradable biological materials. In a further embodiment, the reinforcing elements of the reinforced biodegradable stent comprise magnesium or magnesium alloy.

In some embodiments, the base material of the reinforced biodegradable stent is a biodegradable material selected from a group consisting of collagen, gelatin, elastin or tropoelastin, chitosan, NOCC, low MW chitosan, fibrin glue, biological sealant, chitosan-alginate complex, chitosan-glycerol complex, and combination thereof. In one embodiment, the base material of the reinforced biodegradable stent is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof. In another embodiment, the base material of the reinforced biodegradable stent is crosslinked with a means for crosslinking the material, the means comprising exposing the material to ultraviolet or gamma irradiation, dehydrothermal treatment, tris(hydroxymethyl)phosphine, ascorbate-copper, glucose-lysine or photo-oxidizers. In still another embodiment, the base material of the reinforced biodegradable stent is a biodegradable material selected from a group consisting of poly(L-lactic acid), polyglycolic acid, poly(D,L-lactide-co-glycolide), polycaprolactone, poly(ester amides), mixture thereof, and co-polymers thereof.

In some embodiments, the reinforced biodegradable stent is configured to be a tubular shape, a meshed tubular shape, or a non tubular shape prior to being loaded in a delivery apparatus.

In some embodiments, the reinforced biodegradable stent further comprises at least one bioactive agent. In one embodiment, the reinforced biodegradable stent further comprises one or more imaging materials to render the stent visible under either magnetic resonance imaging or x-ray based fluoroscopy procedure.

Some aspects of the invention relate to a method of treating atherosclerosis of a patient comprising: providing a biodegradable vascular stent comprising a composite material, wherein the composite material comprises a crosslinkable base material and a plurality of reinforcing elements; crosslinking the base material; and delivering the stent to the atherosclerosis for treating the atherosclerosis. In a further embodiment, the reinforcing elements are characterized in that a first Young's modulus of at least a portion of the elements is greater than a second Young's modulus of the base material. In a further embodiment, the vascular stent further comprises at least one bioactive agent.

Some aspects of the invention relate to a biodegradable stent comprising a luminal surface portion with a second degree of crosslink, an outer surface portion with a first degree of crosslink, and a wall between the luminal and outer surface portions, wherein the wall comprises a crosslinked material characterized by the first degree of crosslink not less than the second degree of crosslink. In one embodiment, the crosslinked material is a biodegradable material selected from a group consisting of collagen, gelatin, elastin or tropoelastin, chitosan, NOCC, low MW chitosan, fibrin glue, biological sealant, chitosan-alginate complex, chitosan-glycerol complex, and combination thereof. In a further embodiment, the crosslinked material is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof. In another embodiment, the crosslinked material is a biodegradable material selected from a group consisting of polylactic acid (PLA), polyglycolic acid (PGA), poly(D,L-lactide-co-glycolide), polycaprolactone, poly(amides), poly(ester amides), polyhydroxy acids, polyalkanoates, polyanhydrides, polyphosphazenes, polyetheresters, polyesteramides, polyesters, polyorthoesters, co-polymers thereof, and mixture thereof.

In a further embodiment, the stent further comprises at least one bioactive agent. In one preferred embodiment, the bioactive agent is conjugated to a targeting moiety, wherein the targeting moiety is porphyrin, motexafin lutetium, or a non-porphyrin drug facilitator.

Some aspects of the invention relate to a biodegradable stent comprising a luminal surface portion, an outer surface portion, and a wall between the luminal and outer surface portions, wherein the wall comprises at least one bioactive agent conjugated to a targeting moiety. In one embodiment, the wall comprises a crosslinked material characterized by a degree of crosslink gradually increases from the luminal surface portion to the outer surface portion. In another embodiment, the wall comprises a crosslinked material characterized by a first degree of crosslink at the outer surface portion not less than a second degree of crosslink at the luminal surface portion.

Some aspects of the invention relate to a method of treating vascular atherosclerosis comprising: placing a biodegradable stent proximal to the atherosclerosis, wherein the stent comprises at least one bioactive agent; releasing the bioactive agent; and treating the vascular atherosclerosis distal to the stent. In a further embodiment, the method further comprises a step of providing a distal protection by placing a filter apparatus distal to the atherosclerosis for collecting and removing unwanted micro particles. In a further embodiment, the bioactive agent is conjugated to a targeting moiety.

Some aspects of the invention are to provide a biodegradable implant comprising: a body made of a biodegradable material, wherein the body has an external surface; and a barrier material coated on at least a portion of the surface. In a further embodiment, the biodegradable material has a first degree of biodegradation and the barrier material has a second degree of biodegradation, the first degree of biodegradation being higher than the second degree of biodegradation. In a further embodiment, the biodegradable material has a first swelling ratio and the barrier material has a second swelling ratio, the first swelling ratio being higher than the second swelling ratio. In a further embodiment, the biodegradable material is a crosslinked material, wherein the implant may further comprise at least one bioactive agent. The term “diffiusion-restricting barrier material” is meant to refer herein to a material with reduced or restricted diffusivity for certain molecules.

Some aspects of the invention are to provide a biodegradable vascular stent comprising: a body made of a biodegradable material, wherein the body has an external surface; and a diffusion-restricting barrier material loaded on at least a portion of the body or coated on at least a portion of the surface of the body. In a further embodiment, the barrier material is a hydrophobic material, a hydrophobic chitosan, or a material selected from a group consisting of poly(L-lactic acid), polyglycolic acid, poly(D,L-lactide-co-glycolide), polycaprolactone, poly(ester amides), mixture thereof, and co-polymers thereof.

Some aspects of the invention are to provide a method of treating vascular atherosclerosis comprising: a) providing a biodegradable stent, wherein the stent comprises at least one imaging material to render the implant visible under either magnetic resonance imaging or x-ray based fluoroscopy procedure; and b) deploying the stent at about the vascular atherosclerosis using the magnetic resonance imaging or the x-ray fluoroscopy guidance.

Some aspects of the invention are to provide a biodegradable vascular stent for treating atherosclerosis comprising a composite material, wherein the composite material comprises a non-metallic base material and means for enhancing the radial strength of the stent, wherein the means for enhancing the radial strength of the stent may comprise a textured surface on at least a portion of the composite material or a raised elongate rib along the long axis of at least one open-ring element of the vascular stent.

Some aspects of the invention relate to a biodegradable vascular stent for treating atherosclerosis in a blood vessel comprising a stent body made of a biodegradable material, wherein the stent body is configured to be a polygon shaped sheet before being implanted in the blood vessel. In a further embodiment, the stent body having an internal surface, an external surface, and a plurality of openings between the internal and external surfaces, wherein the stent body is configured to be a non-tubular shape in a first position prior to being loaded in a delivery apparatus for delivering the stent to the blood vessel, and is configured to be a tubular-like shape after being placed in the blood vessel. In one embodiment, the stent is a quadrilateral polygon, wherein the stent may be a rectangular or square polygon. In another embodiment, a periphery of the openings is being configured to be continuously smooth without any cut or sharp point.

In one embodiment, the biodegradable vascular stent configured to be a polygon shaped sheet comprises at least one imaging material to render the implant visible under either magnetic resonance imaging or x-ray based fluoroscopy procedure. In another embodiment,

Some aspects of the invention relate to a biodegradable implant comprising a biodegradable material, wherein the implant is sized and configured to be a polygon shape and has an internal surface, an external surface, and a plurality of openings between the internal and external surfaces, the implant body being configured to be an essentially non-tubular shape before being implanted. In one embodiment, the implant is a rectangular or square polygon.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 shows chemical structures of glutaraldehyde and genipin that are used in the chemical treatment examples of the current disclosure.

FIG. 2A shows an iridoid glycoside present in fruits of Gardenia jasmindides Ellis (Structure I).

FIG. 2B shows a parent compound geniposide (Structure II) from which genipin is derived.

FIG. 3 shows a proposed crosslinking mechanism for a crosslinker, glutaraldehyde (GA) with collagen intermolecularly and/or intramolecularly.

FIG. 4A shows a proposed reaction mechanism between genipin and an amino group of a reactant, including collagen or certain type of drug of the present invention.

FIG. 4B shows a proposed crosslinking mechanism for a crosslinker, genipin (GP) with collagen intermolecularly and/or intramolecularly.

FIG. 5 shows a schematic illustration for genipin to crosslink an amino-containing collagen and an amino-containing drug.

FIG. 6 shows an illustrated example of a cross-sectional view for a vascular stent coated with drug-containing collagen crosslinked with genipin according to the principles of the present invention.

FIG. 7 shows one embodiment of a cross-sectional view for a vascular stent coated with drug-containing collagen layers that are crosslinked with genipin.

FIG. 8 shows another embodiment of a longitudinal view for a vascular stent coated with drug-containing collagen layers that are crosslinked with genipin.

FIG. 9 shows a biodegradable stent comprising a first supporting zone that comprises at least a portion of continuous circumference of the stent and a second therapeutic zone.

FIG. 10 shows an enlarged view of the biodegradable stent, section I-I of FIG. 9, showing the interface of the first supporting zone and the second therapeutic zone.

FIG. 11 shows a perspective view of placing the biodegradable stent of the invention at the vulnerable plaque of a patient.

FIG. 12 shows one embodiment of a spiral (helical) biodegradable stent according to the principles of the invention.

FIG. 13 shows one embodiment of a double helical biodegradable stent according to the principles of the invention.

FIG. 14A shows one embodiment of an open-ring biodegradable stent at a pre-deployment stage.

FIG. 14B shows one embodiment of an open-ring biodegradable stent at a post-deployment stage.

FIG. 15 shows another embodiment of an open-ring biodegradable stent according to the principles of the invention.

FIG. 16 shows a further embodiment of an open-ring biodegradable stent according to the principles of the invention.

FIG. 17 shows still another embodiment of an open-ring biodegradable stent with spirally oriented open pattern according to the principles of the invention.

FIG. 18 shows one embodiment of an interlocking open-ring biodegradable stent according to the principles of the invention.

FIG. 19A shows one embodiment of a stent not in a tubular shape before implantation according to the principles of the invention.

FIG. 19B shows the curved stent of FIG. 19A within a catheter sheath during a stent deployment phase.

FIG. 19C shows another embodiment of a stent not in a tubular shape before implantation according to the principles of the invention.

FIG. 19D shows a non-tubular shaped stent of FIG. 19C having at least one curved open-ring end.

FIG. 20 shows one embodiment of a delivery apparatus for deploying a biodegradable stent.

FIG. 21 shows another embodiment of a delivery apparatus for deploying a biodegradable stent.

FIG. 22A shows one embodiment of an open-ring biodegradable stent with ring enforcement means for enhancing crushing resistance.

FIG. 22B shows a first detailed ring enforcement means, section II-II of FIG. 22A, showing a raised elongate rib along one ring element.

FIG. 22C shows a second detailed ring enforcement means, section III-III of FIG. 22A, showing a textured surface on the surface of one ring element.

FIG. 23A shows one embodiment of a biodegradable cardiovascular sheet stent in a first position before implantation.

FIG. 23B shows the biodegradable cardiovascular sheet stent of FIG. 23A in a second position being loaded in a delivery apparatus before implantation.

FIG. 24 shows one embodiment of deploying a sheet stent using a delivery apparatus according to the principles of the present invention.

### DETAILED DESCRIPTION OF EXEMPLARY EMBODIMENTS

The following detailed description is of the best presently contemplated modes of carrying out the invention. This description is not to be taken in a limiting sense, but is made merely for the purpose of illustrating general principles of embodiments of the invention.

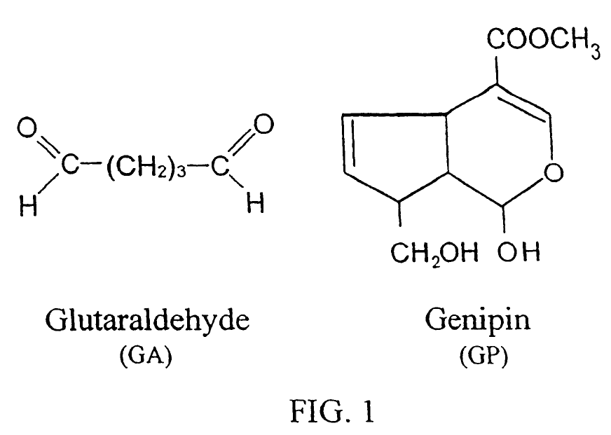

“Genipin” in this invention is meant to refer to the naturally occurring compound as shown in FIG. 1 and its derivatives, analog, stereoisomers and mixtures thereof.

“Crosslinking agent” is meant herein to indicate a chemical agent that could crosslink two molecules, such as formaldehyde, glutaraldehyde, dialdehyde starch, glyceraldehydes, cyanamide, diimides, diisocyanates, dimethyl adipimidate, carbodiimide, genipin, proanthocyanidin, reuterin, and epoxy compound.

“Biological material” is herein meant to refer to collagen (collagen extract, soluble collagen, collagen solution, or other type of collagen), elastin or tropoelastin, gelatin, fibrin glue, biological sealant, chitosan (including N, O, carboxylmethyl chitosan), chitosan-containing and other collagen-containing biological material. In a further embodiment, the “biological material” is intended synonymous to “biopolymers” herein. For an alternate aspect of the present invention, the biological material is also meant to indicate a solidifiable biological substrate comprising at least a crosslinkable functional group, such as amino group or the like.

A “biological implant” refers to a medical device which is inserted into, or grafted onto, bodily tissue to remain for a period of time, such as an extended-release drug delivery device, drug-eluting stent, vascular or skin graft, or orthopedic prosthesis, such as bone, ligament, tendon, cartilage, and muscle.

In particular, the crosslinked collagen-drug device or compound with drug slow release capability may be suitable in treating atherosclerosis and for other therapeutic applications. In one aspect of the invention, it is provided a biodegradable medical device comprising a plurality layers or zones, each with at least one bioactive agent and at least one biological material. The biodegradable medical device is thereafter crosslinked with a crosslinking agent. “Biodegradable” and “bioabsorbable” are interchangeable in meaning herein in this disclosure. In one embodiment, the layers and zones are configured and arranged, in combination, radially, circumferentially and longitudinally.

“Drug” in this invention is meant to broadly refer to a chemical molecule(s), biological molecule(s) or bioactive agent providing a therapeutic, diagnostic, or prophylactic effect in vivo. “Drug” and “bioactive agent” (interchangeable in meaning herein in this disclosure) may comprise, but not limited to, synthetic chemicals, biotechnology-derived molecules, herbs, cells, genes, growth factors, health food and/or alternate medicines. In the present invention, the terms “drug” and “bioactive agent” are used interchangeably

A blood vessel is generally consisted of a support structure for transporting blood and a luminal blood-contacting surface lined with a layer of endothelial cells. On a denuded vessel surface, endothelialization, which involves the migration of endothelial cells from adjacent tissue onto the denuded luminal surface, can occur as a part of the healing process. Unfortunately, self-endothelialization occurs to only a limited degree and the limited endothelialization that does occur takes place slowly. To promote the rapid formation of an endothelial lining, endothelial cells can be seeded or loaded onto an implant, for example, a drug-eluting device of the present invention, before the implant is placed in the recipient. When the implant is placed in the recipient and exposed to physiologic blood flow, a portion of the endothelial cells at the device surface starts the process of endothelialization while another portion of the endothelial cells is slowly released to the device surface having delayed endothelialization.

The “biological substance” is herein intended to mean a substance made of drug-containing biological material that is, in one preferred embodiment, solidifiable upon change of environmental condition(s) and is biocompatible after being crosslinked with a crosslinker, such as genipin, epoxy compounds, dialdehyde starch, glutaraldehyde, formaldehyde, dimethyl adipimidate, carbodiimide, proanthocyanidin, or the like. Some aspects of the invention provide a crosslinked biodegradable stent or implant comprising at least one layer or zone of biological material, the biological material comprising at least one bioactive agent and being crosslinked with a means for crosslinking the biological material.

Preparation and Properties of Genipin

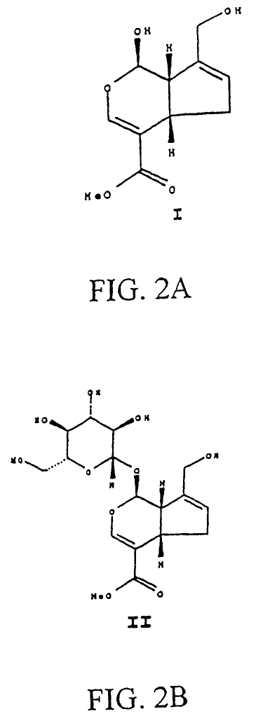

Genipin, shown in Structure I of FIG. 2A, is an iridoid glycoside present in fruits (Gardenia jasmindides Ellis). It may be obtained from the parent compound geniposide, Structure II (FIG. 2B), which may be isolated from natural sources as described in elsewhere (Sung H W et al., in Biomaterials and Drug Delivery toward New Millennium, Eds K D Park et al., Han Rin Won Publishing Co., pp. 623-632, (2000)). Genipin, the aglycone of geniposide, may be prepared from the latter by oxidation followed by reduction and hydrolysis or by enzymatic hydrolysis. Alternatively, racemic genipin may be prepared synthetically. Although Structure I shows the natural configuration of genipin, any stereoisomer or mixture of stereoisomers of genipin as shown later may be used as a crosslinking reagent, in accordance with the present invention.

Genipin has a low acute toxicity, with LD₅₀ i.v. 382 mg/k in mice. It is therefore much less toxic than glutaraldehyde and many other commonly used synthetic crosslinking reagents. As described below, genipin is shown to be an effective crosslinking agent for treatment of biological materials intended for in vivo biomedical applications, such as prostheses and other implants, wound dressings, and substitutes.

It is one object of the present invention to provide a drug-collagen-genipin and/or drug-chitosan-genipin compound that is loaded onto the periphery of a cardiovascular stent enabling drug slow-release to the surrounding tissue, or to the lumen of the bodily cavity. In one preferred embodiment, the compound is loaded onto the outer periphery of the stent enabling drug slow-release to the surrounding tissue. In another embodiment, the compound is fabricated as a stent enabling drug slow-release to the surrounding tissue.

Previously, Chang in U.S. Pat. No. 5,929,038 discloses a method for treating hepatitis B viral infection with an iridoid compound of a general formula containing a six-member hydrocarbon ring sharing with one common bondage of a five-member hydrocarbon ring. Further, Moon et al. in U.S. Pat. No. 6,162,826 and No. 6,262,083 discloses genipin derivatives having anti hepatitis B virus activity and liver protection activity. All of which three aforementioned patents are incorporated herein by reference. The teachings of these patents do not disclose preparing tissue/device with scaffolds or collagen matrix with desirable porosity for use in tissue engineering, wherein the raw material source for tissue engineering is chemically modified by genipin, genipin derivatives or its analog with acceptably minimal cytotoxicity.

The genipin derivatives and/or genipin analog may have the following chemical formulas (Formula 1 to Formula 4):

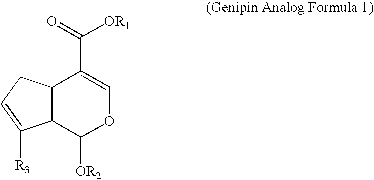

in which

R₁ represents lower alkyl;

R₂ represents lower alkyl, pyridylcarbonyl, benzyl or benzoyl;

R₃ represents formyl, hydroxymethyl, azidomethyl, 1-hydroxyethyl, acetyl, methyl, hydroxy, pyridylcarbonyl, cyclopropyl, aminomethyl substituted or unsubstituted by (1,3-benzodioxolan-5-yl)carbonyl or 3,4,5-trimethoxybenzoyl, 1,3-benzodioxolan-5-yl, ureidomethyl substituted or unsubstituted by 3,4,5-trimethoxyphenyl or 2-chloro-6-methyl-3-pyridyl, thiomethyl substituted or unsubstituted by acetyl or 2-acetylamino2-ethoxycarbonyethyl, oxymethyl substituted or unsubstituted by benzoyl, pyridylcarbonyl or 3,4,5-trimethoxybenzoyl;

provided that R₃ is not methyl formyl, hydroxymethyl, acetyl, methylaminomethyl, acetylthiomethyl, benzoyloxymethyl or pyridylcarbonyloxymethyl when R₁ is methyl, and

its pharmaceutically acceptable salts, or stereoisomers.

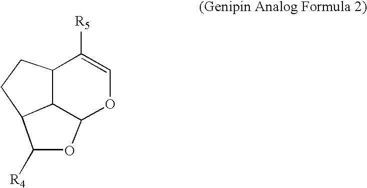

in which

R₄ represents lower alkoxy, benzyloxy, benzoyloxy, phenylthio, C₁˜C₁₂ alkanyloxy substituted or unsubstituted by t-butyl, phenyl, phenoxy, pyridyl or thienyl;

R₅ represents methoxycarbonyl, formyl, hydroxyiminomethyl, methoxyimino-methyl, hydroxymethyl, phenylthiomethyl or acetylthiomethyl;

provided that R₅ is not methoxycarbonyl when R₁₄ is acetyloxy; and

its pharmaceutically acceptable salts, or stereoisomers.

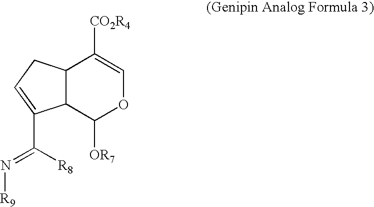

R₆ represents hydrogen atom, lower alkyl or alkalimetal;

R₇ represents lower alkyl or benzyl;

R₈ represents hydrogen atom or lower alkyl;

R₉ represents hydroxy, lower alkoxy, benzyloxy, nicotinoyloxy, isonicotinoyloxy, 2-pyridylmethoxy or hydroxycarbonylmethoxy;

provided that R₉ is not hydroxy or methoxy when R₆ is methyl and R₈ is hydrogen atom; and

its pharmaceutically acceptable salts, or stereoisomers.

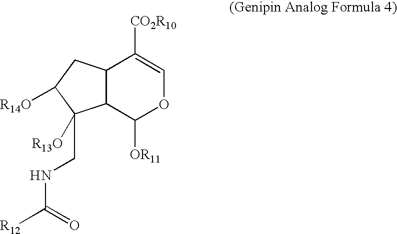

in which

R₁₀ represents lower alkyl;

R₁₁ represents lower alkyl or benzyl;

R₁₂ represents lower alkyl, pyridyl substituted or unsubstituted by halogen, pyridylamino substituted or unsubstituted by lower alkyl or halogen, 1,3-benzodioxolanyl;

R₁₃ and R₁₄ each independently represent a hydrogen atom or join together to form isopropylidene; and

its pharmaceutically acceptable salts, or stereoisomers.

Kyogoku et al. in U.S. Pat. No. 5,037,664, U.S. Pat. No. 5,270,446, and EP 0366998, the entire contents of all three being incorporated herein by reference, teach the crosslinking of amino group containing compounds with genipin and the crosslinking of genipin with chitosan. They also teach the crosslinking of iridoid compounds with proteins which can be vegetable, animal (collagen, gelatin) or microbial origin. However, they do not teach loading drug onto a collagen-containing biological material crosslinked with genipin as biocompatible drug carriers for drug sustained release.

Toyama et al. in U.S. Pat. No. 4,247,698, the entire contents of which are incorporated herein by reference, discloses a particular genipin analog group with red coloring characterized by substituting R₁═H or CH₃ in the Genipin Analog Formula I (paragraph 0091) of the present invention. This particular genipin analog group is generally called aglycon geniposidic acid.

Smith in U.S. Pat. No. 5,322,935, incorporated herein by reference in its entirety, teaches the crosslinking of chitosan polymers and then further crosslinking again with covalent crosslinking agents like glutaraldehyde. Smith, however, does not teach loading drug onto a chitosan-containing biological material crosslinked with genipin as biocompatible drug carriers for drug slow-release.

Noishiki et al. in U.S. Pat. No. 4,806,595 discloses a tissue treatment method by a crosslinking agent, polyepoxy compounds. Collagens used in that patent include an insoluble collagen, a soluble collagen, an atelocollagen prepared by removing telopeptides on the collagen molecule terminus using protease other than collagenase, a chemically modified collagen obtained by succinylation or esterification of above-described collagens, a collagen derivative such as gelatin, a polypeptide obtained by hydrolysis of collagen, and a natural collagen present in natural tissue (ureter, blood vessel, pericardium, heart valve, etc.) The Noishiki et al. patent is incorporated herein by reference. “Biological material” in the present invention is additionally used herein to refer to the above-mentioned collagen, collagen species, collagen in natural tissue, and collagen in a biological implant preform that are shapeable and/or solidifiable.

Voytik-Harbin et al. in U.S. Pat. No. 6,264,992 discloses submucosa as a growth substrate for cells. More particularly, the submucosa is enzymatically digested and gelled to form a shape retaining gel matrix suitable for inducing cell proliferation and growth both in vivo and in vitro. The Voytik-Harbin et al. patent is incorporated herein by reference. Biological material, additionally including submucosa, that is chemically modified or treated by genipin or other crosslinker of the present invention may serve as a shapeable raw material for making a biological substance adapted for inducing cell proliferation and ingrowth, but also resisting enzymatic degradation, both in vivo and in vitro. In a further aspect of the present invention, drug is loaded with submucosa biological material and crosslinked with a crosslinker, such as genipin.

Cook et al. in U.S. Pat. No. 6,206,931 discloses a graft prosthesis material including a purified, collagen-based matrix structure removed from a submucosa tissue source, wherein the submucosa tissue source is purified by disinfection and removal steps to deactivate and remove contaminants. The Cook et al. patent is incorporated herein by reference. Similarly, a collagen-based matrix structure, also known as a part of “biological material” in this disclosure, may serve as a biomaterial adapted for medical device use after chemical modification by genipin of the present invention.

Several disadvantages are associated with the currently available technology. First, the prior art teaches collagen or chitosan in drug delivery application without suitable crosslinkage. The drug within collagen or chitosan matrix may tend to leach out in a short period of time because of no crosslinked barriers surrounding the drug. Another prior art teaches crosslinked collagen or chitosan without drug slow-release properties. It is essential that drug is appropriately loaded within collagen or chitosan before the drug-containing collagen/chitosan is crosslinked enabling drug slow-release. Therefore, even if the two afore-mentioned prior arts were to be combined in a conventional manner, the combination would not show all of the novel physical feature and unexpected results of the present invention.

In a co-pending patent application Ser. No. 10/282,852 filed Oct. 29, 2002, entitled “Reuterin and methods of use”, it is disclosed that reuterin (β-hydroxypropionaldehyde) as a naturally occurring crosslinking agent can react with the free amino groups of biological material of the present invention.

Collagen-Drug-Genipin Compound

In one embodiment of the present invention, it is disclosed that a method for treating tissue of a patient comprising, in combination, providing a drug-containing biological material to be shaped as a medical device, chemically treating the drug-containing biological material with a crosslinking agent, and delivering the medical device to a target tissue for releasing the drug and treating the tissue. The compound (such as collagen-drug-genipin compound, the chitosan-drug-genipin compound, or combination thereof) and methods of manufacture as disclosed and supported by the following examples produce new and unexpected results and hence are unobvious from the prior art. The medical device can be a stent, a non-stent implant or prosthesis for the intended drug slow release. In a preferred aspect, the stent application with the compound (such as collagen-drug-genipin compound, the chitosan-drug-genipin compound, or combination thereof) comprises medical use in lymphatic vessel, gastrointestinal tract (including the various ducts such as hepatic duct, bile duct, pancreatic duct, etc.), urinary tract (ureter, urethra, etc.), and reproductive tract (i.e., uterine tube, etc.). In one aspect, the non-stent implant may comprise annuloplasty rings, heart valve prostheses, venous valve bioprostheses, orthopedic implants, dental implants, ophthalmology implants, cardiovascular implants, and cerebral implants. In another aspect of the present invention, the target tissue may comprise vulnerable plaque, atherosclerotic plaque, tumor or cancer, brain tissue, vascular vessel or tissue, orthopedic tissue, ophthalmology tissue or the like. The vulnerable plaque is the atherosclerotic plaque that is vulnerably prone to rupture in a patient.

In another embodiment of the present invention, it is disclosed a biological substance for treating tissue of a patient with drug slow release, wherein the biological substance is made of drug-containing biological material that may be solidifiable upon change of environmental condition(s) and is biocompatible after being crosslinked with a crosslinker, such as genipin, epoxy compounds, dialdehyde starch, dimethyl adipimidate, carbodiimide, glutaraldehyde, or the like.

In still another embodiment of the present invention, it is disclosed that a method for treating tissue of a patient comprising, in combination, mixing a drug with a biological material, pre-forming the drug containing biological material as a medical device, chemically treating the pre-formed biological material with a crosslinking agent, and delivering the crosslinked biological material to a lesion site for treating the tissue. In one alternate embodiment, the method further comprises a step of solidifying the drug-containing biological material.

It is some aspect of the present invention that the method may further comprise chemically linking the drug with the biological material through a crosslinker, wherein the drug comprises at least a crosslinkable functional group, for example, an amino group.

It is a further aspect of the present invention to provide a method for treating vascular restenosis comprising, in combination, providing a drug-containing biological material shaped and configured as a medical device, chemically treating the device with a crosslinking agent, and delivering the medical device to a vascular restenosis site for treating the vascular restenosis. In one embodiment, the method further comprises a step of solidifying the drug-containing biological material.

Drug for Use in Collagen-Drug-Genipin Compound

The drugs used in the current generation drug eluting cardiovascular stents include two major mechanisms: cytotoxic and cytostatic. Some aspects of the invention relating to the drugs used in collagen-drug-genipin compound from the category of cytotoxic mechanism comprise actinomycin D, paclitaxel, vincristin, methotrexate, and angiopeptin. Some aspects of the invention relating to the drugs used in collagen-drug-genipin compound from the category of cytostatic mechanism comprise batimastat, halofuginone, sirolimus, tacrolimus, everolimus, tranilast, dexamethasone, and mycophenolic acid (MPA). Some aspects of the present invention provide a bioactive agent in a bioactive agent-eluting device, wherein the bioactive agent is selected from a group consisting of actinomycin D, paclitaxel, vincristin, methotrexate, and angiopeptin, batimastat, halofuginone, sirolimus, tacrolimus, everolimus, tranilast, dexamethasone, and mycophenolic acid.

Everolimus with molecular weight of 958 (a chemical formula of C₅₃H₈₃NO₁₄) is poorly soluble in water and is a novel proliferation inhibitor. There is no clear upper therapeutic limit of everolimus. However, thrombocytopenia occurs at a rate of 17% at everolimus trough serum concentrations above 7.8 ng/ml in renal transplant recipients (Expert Opin Investig Drugs 2002; 11(12):1845-1857). In a patient, everolimus binds to cytosolic immunophyllin FKBP12 to inhibit growth factor-driven cell proliferation. Everolimus has shown promising results in animal studies, demonstrating a 50% reduction of neointimal proliferation compared with a control bare metal stent.

Straub et al. in U.S. Pat. No. 6,395,300 discloses a wide variety of drugs that are useful in the methods and compositions described herein, the entire contents of which, including a variety of drugs, are incorporated herein by reference. Drugs contemplated for use in the compositions described in U.S. Pat. No. 6,395,300 and herein disclosed include the following categories and examples of drugs and alternative forms of these drugs such as alternative salt forms, free acid forms, free base forms, and hydrates:

analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate);

antiasthamatics (e.g., ketotifen and traxanox);

antibiotics (e.g., neomycin, streptomycin, chloramphenicol, cephalosporin, ampicillin, penicillin, tetracycline, and ciprofloxacin);

antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline);

antidiabetics (e.g., biguanides and sulfonylurea derivatives);

antifungal agents (e.g., griseofulvin, ketoconazole, itraconizole, amphotericin B, nystatin, and candicidin);

antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);

anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone);

antineoplastics (e.g., cyclophosphamide, actinomycin, bleomycin, daunorubicin, doxorubicin hydrochloride, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, piposulfan,);

antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);

immunosuppressive agents (e.g., cyclosporine, azathioprine, mizoribine, and FK506 (tacrolimus));

antimigraine agents (e.g., ergotamine, propanolol, isometheptene mucate, and dichloralphenazone);

sedatives/hypnotics (e.g., barbiturates such as pentobarbital, pentobarbital, and secobarbital; and benzodiazapines such as flurazepam hydrochloride, triazolam, and midazolam);

antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate);

antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine);

antimanic agents (e.g., lithium carbonate);

antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine);

antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium);

antigout agents (e.g., colchicine, and allopurinol);

anticoagulants (e.g., heparin, heparin sodium, and warfarin sodium);

thrombolytic agents (e.g., urokinase, streptokinase, and alteplase);

antifibrinolytic agents (e.g., aminocaproic acid);

hemorheologic agents (e.g., pentoxifylline);

antiplatelet agents (e.g., aspirin);

anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione);

antiparkinson agents (e.g., ethosuximide);

antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorphenirarnine maleate, methdilazine, and);

agents useful for calcium regulation (e.g., calcitonin, and parathyroid hormone);

antibacterial agents (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate);

antiviral agents (e.g., interferon alpha, beta or gamma, zidovudine, amantadine hydrochloride, ribavirin, and acyclovir);

antimicrobials (e.g., cephalosporins such as cefazolin sodium, cephradine, cefaclor, cephapirin sodium, ceftizoxime sodium, cefoperazone sodium, cefotetan disodium, cefuroxime azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin);

anti-infectives (e.g., GM-CSF);

bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate;

steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);

hypoglycemic agents (e.g., human insulin, purified beef insulin, purified pork insulin, glyburide, chlorpropamide, glipizide, tolbutarnide, and tolazamide);

hypolipidemic agents (e.g., clofibrate, dextrothyroxine sodium, probucol, pravastitin, atorvastatin, lovastatin, and niacin);

proteins (e.g., DNase, alginase, superoxide dismutase, and lipase);

nucleic acids (e.g., sense or anti-sense nucleic acids encoding any therapeutically useful protein, including any of the proteins described herein);

agents useful for erythropoiesis stimulation (e.g., erythropoietin);

antiulcer/antireflux agents (e.g., famotidine, cimetidine, and ranitidine hydrochloride);

antinauseants/antiemetics (e.g., meclizine hydrochloride, nabilone, prochlorperazine, dimenhydrinate, promethazine hydrochloride, thiethylperazine, and scopolamine);

as well as other drugs useful in the compositions and methods described herein include mitotane, halonitrosoureas, anthrocyclines, ellipticine, ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.

Preferred drugs useful in the present invention may include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, clobustasol, diflucortolone, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

Specific non-limiting examples of some drugs that fall under the above categories include paclitaxel, docetaxel and derivatives, epothilones, nitric oxide release agents, heparin, aspirin, coumadin, PPACK, hirudin, polypeptide from statins, angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents, and dexamethasone, tacrolimus, actinomycin-D, batimastat etc.

Sirolimus is a naturally occurring macrolide antibiotic produced by the fungus Streptomyces found in Easter Island. It was discovered by Wyeth-Ayerst in 1974 while screening fermentation products. Sirolimus with molecular weight of 916 (a chemical formula of C₅₁H₇₉NO₁₃) is non-water soluble and is a potential inhibitor of cytokine and growth factor mediated cell proliferation. FDA approved its use as oral immunosuppressive agents with a formulation of 2 to 5 mg/dose. The suggested drug-eluting efficacy is about 140 micrograms/cm², 95% drug release at 90 days and 30% drug-to-polymer ratio.

In some aspect of the present invention, the drug (also referred as a bioactive agent) may broadly comprise, but not limited to, synthetic chemicals, biotechnology-derived molecules, herbs, health food, extracts, and/or alternate medicines; for example, including allicin and its corresponding garlic extract, ginsenosides (for example, Rg1, Re, or thelike) and the corresponding ginseng extract, flavone/terpene lactone and the corresponding ginkgo biloba extract, glycyrrhetinic acid and the corresponding licorice extract, and polyphenol/proanthocyanides and the corresponding grape seed extract.

While the preventive and treatment properties of the foregoing therapeutic substances, agents, drugs, or bioactive agents are well known to those having ordinary skill in the art, the substances or agents are provided by way of example and are not meant to be limiting. Other therapeutic substances are equally applicable for use with the disclosed methods, devices, and compositions.

In the present invention, the terms “crosslinking”, “fixation”, “chemical modification”, and “chemical treatment” for tissue are used interchangeably.

FIG. 1 shows chemical structures of glutaraldehyde and genipin that are used in the chemical treatment examples of the current disclosure. Other crosslink agents may equally be applicable for collagen-drug-genipin and/or chitosan-drug-genipin compound disclosed herein.

Other than genipin and glutaraldehyde, the crosslinking agent that may be used in chemical treatment of the present invention may include formaldehyde, dialdehyde starch, glyceraldehydes, cyanamide, diimides, diisocyanates, dimethyl adipimidate, carbodiimide, and epoxy compound.

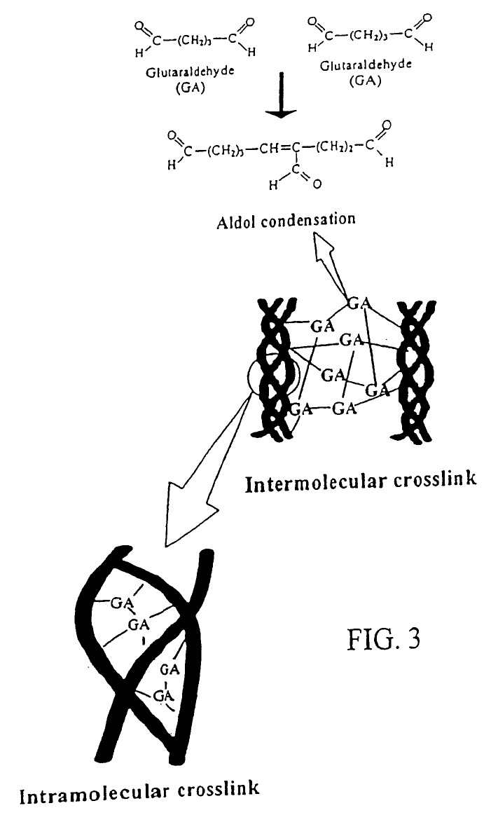

FIG. 3 shows a proposed crosslinking mechanism for a crosslinker, glutaraldehyde (GA) with collagen intermolecularly and/or intramolecularly.

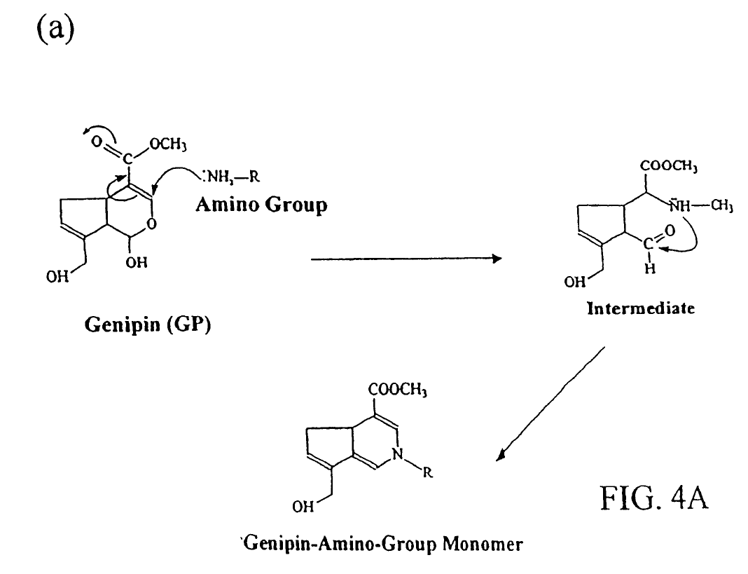

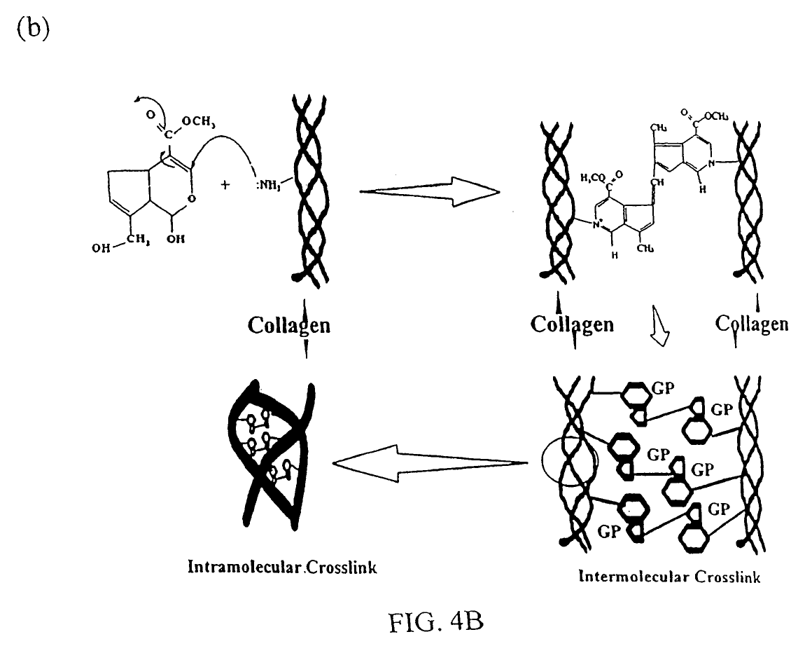

FIG. 4A shows a proposed reaction mechanism between genipin and an amino group of a reactant, including collagen or certain type of drug of the present invention, while FIG. 4B shows a proposed crosslinking mechanism for a crosslinker, genipin (GP) with collagen intermolecularly and/or intramolecularly.

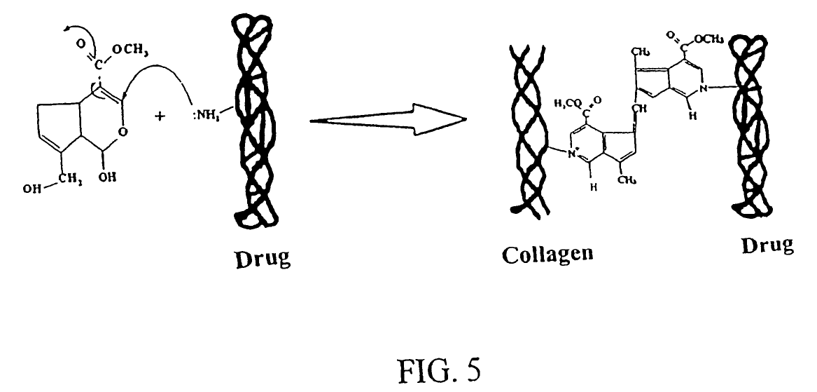

FIG. 5 is a schematic illustration for genipin to crosslink an amino-containing collagen and an amino-containing drug. It is also conceivable for a crosslinker, such as genipin to link an amine-containing substrate and an amino-containing drug. An example of amine-containing substrate is polyurethane and the like.

Glutaraldehyde Crosslinking

Glutaraldehyde has been used extensively as a crosslinking agent for fixing biologic tissues. By means of its aldehyde functional groups, glutaraldehyde reacts primarily with the ε-amino groups of lysyl or hydroxylysyl residues within biologic tissues. The mechanism of fixation of biologic tissues or biologic matrix with glutaraldehyde can be found elsewhere. Polymerization of glutaraldehyde molecules in aqueous solution with observable reductions in free aldehyde have been reported previously (Nimni M E et al. in Nimni M E, editor. COLLAGEN. Vol. III. Boca Raton (Fla.); CRC Press 1998. pp. 1-38). In polymerization the aldehyde functional groups of 2 glutaraldehyde molecules may undergo an aldol condensation (FIG. 3). With glutaraldehyde polymerization, subsequent to fixation, a network crosslinking structure could conceivably be created intramolecularly and intermolecularly within collagen fibers (FIG. 3).

It is conceivable that a substance (for example, a drug) having an amine or amino functional group may react with glutaraldehyde as illustrated above. By combining collagen, glutaraldehyde and a drug having an amine or amino group, the crosslinked compound may link collagen to the drug via glutaraldehyde as a crosslinker.

Crosslinking of a Polymer Having an Amine Group

Several biocompatible plastic polymers or synthetic polymers have one or more amine group in their chemical structures, for example poly(amides) or poly(ester amides). The amine group may become reactive toward a crosslinker, such as glutaraldehyde, genipin or epoxy compounds. Therefore, it is conceivable that by combining a polymer having an amine group, glutaraldehyde and a drug having at least an amine or amino group, the crosslinked compound may have the polymer linked to the drug via glutaraldehyde as a crosslinker. Other crosslinkers are also applicable.

Genipin Crosslinking

It was found by Sung H W (Biomaterials 1999; 20:1759-72) that genipin can react with the free amino groups of lysine, hydroxylysine, or arginine residues within biologic tissues. A prior study reports that the structures of the intermediates, leading to a blue pigment produced from genipin and methylamine, the simplest primary amine. The mechanism was suggested that the genipin-methylamine monomer is formed through a nucleophilic attack by methylamine on the olefinic carbon at C-3 of genipin, followed by opening of the dihydropyran ring and attack by the secondary amino group on the resulting aldehyde group (FIG. 4A). The blue-pigment was thought formed through oxygen radical-induced polymerization and dehydrogenation of several intermediary pigments.

As disclosed by Sung H W (J Thorac Cardiovasc Surg 2001; 122:1208-1218), the simplest component in the blue pigment was a 1:1 adduct. It was suggested that genipin reacts spontaneously with an amino acid to form a nitrogen iridoid, which undergoes dehydration to form an aromatic monomer. Dimerization occurs at the second stage, perhaps by means of radical reaction. The results suggest that genipin may form intramolecular and intermolecular crosslinks with cyclic structure within collagen fibers in biologic tissue (FIG. 4B) or solidifiable collagen-containing biological material.

It is disclosed herein that genipin is capable of reacting with a drug having an amine or amino group. By combining collagen (or a biological material or matrix), genipin and the drug having an amine or amino group, the crosslinked compound may have collagen linked to the drug via genipin as a bridge crosslinker (FIG. 5).

As disclosed and outlined in the co-pending patent application Ser. No. 10/067,130 filed Feb. 4, 2002, entitled “Acellular biological material chemically treated with genipin” by one of the present inventors, the degrees in inflammatory reaction in the animal studies for the genipin-fixed cellular and acellular tissue were significantly less than their glutaraldehyde-fixed counterparts. Additionally, it was noted that the inflammatory reactions for the glutaraldehyde-fixed cellular and acellular tissue lasted significantly longer than their genipin-fixed counterparts. These findings indicate that the biocompatibility of the genipin-fixed cellular and acellular tissue is superior to the glutaraldehyde-fixed cellular and acellular tissue. It is hypothesized that the lower inflammatory reactions observed for the genipin-fixed cellular and acellular tissue may be due to the lower cytotoxicity of their remaining residues, as compared to the glutaraldehyde-fixed counterparts. In a previous study, it was found that genipin is significantly less cytotoxic than glutaraldehyde (J Biomater Sci Polymer Edn 1999; 10:63-78). The cytotoxicity observed for the glutaraldehyde-fixed cellular and acellular tissue seems to result from a slow leaching out of unreacted glutaraldehyde as well as the reversibility of glutaraldehyde-crosslinking. It was observed that when concentrations above 0.05% glutaraldehyde were used to crosslink materials, a persistent foreign-body reaction occurred (J Biomater Sci Polymer Edn 1999; 10:63-78).

Some aspects of the invention related to genipin-crosslinked gelatin as a drug carrier. Some aspects of the invention related to genipin-crosslinked fibrin glue and/or biological sealant as a drug carrier. In one embodiment, it is provided a method for treating tissue of a patient comprising, in combination, loading a solidifiable drug-containing gelatin (or fibrin glue/biological sealant) onto an apparatus or medical device, solidifying the drug-containing gelatin, chemically treating the gelatin with a crosslinking agent, and delivering the medical device to the tissue for treating the tissue. Gelatin microspheres haven been widely evaluated as a drug carrier. However, gelatin dissolves rather rapidly in aqueous environments, making the use of gelatin difficult for the production of long-term drug delivery systems. Hsing and associates reported that the degradation rate of the genipin-crosslinked microspheres is significantly increased (J Biomed Mater Res 2003; 65A:271-282). U.S. Pat. No. 6,045,570, the entire contents of which are incorporated herein by reference, discloses a non-fibrin biological sealant comprising a gelatin slurry which includes milled gelatin powder, wherein the slurry may include Gelfoam™ powder mixed with a diluent selected from the group consisting of saline and water. In a further disclosure, the biological sealant may thrombin, or calcium.

U.S. Pat. No. 6,624,138, entitled “Drug-loaded Biological Material Chemically Treated with Genipin”, and PCT WO2004/012676 entitled “Drug-loaded Biological Material Chemically Treated with Genipin”, the entire contents of both are incorporated herein by reference, disclose a method for treating tissue of a patient comprising, in combination, mixing a drug with a solidifiable biological material, chemically treating the drug with the biological material with a crosslinking agent, loading the solidifiable drug-containing biological material onto a medical device, solidifying the drug-containing biological material; and delivering the medical device to a target tissue for treating the tissue.

### Example #1

### Chitosan

Dissolve chitosan powder in acetic acid at about pH 4. Chitosan (MW: about 70,000) was purchased from Fluka Chemical Co. of Switzerland. The deacetylation degree of the chitosan used was approximately 85%. Subsequently, adjust the chitosan solution to approximately pH 5.5 (right before it becomes gelled) with NaOH. Add in drug(s) of interest into the chitosan solution. While loading the drug-containing chitosan onto a stent, adjust the environment to pH 7 with NaOH to solidify the chitosan onto the stent. In another embodiment, the drug-containing chitosan can be configured to become a stent or a multiple-layer stent by exposing to an environment of pH 7 to solidify the chitosan stent. The process can be accomplished via a continuous assembly line step by providing gradually increasing pH zones as the device passes by. It is further treated with a crosslinking agent, for example genipin to enhance the biodurability and biocompatibility. Note that the chemical formula for chitosan can be found in Mi F L, Tan Y C, Liang H F, and Sung H W, “In vivo biocompatibility and degradability of a novel injectable-chitosan based implant.” Biomaterials 2002; 23:181-191, and is shown below.

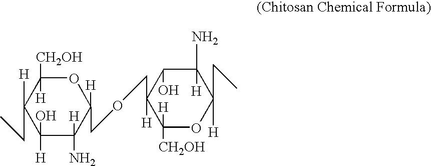

Chitosan is a copolymer of glucosamine and N-acetylglucosamine, derived from the natural polymer chitin, which is commercially available. Chitosan has been reported to be a potentially useful pharmaceutical material because of its good biocompatibility and low toxicity. Some aspects of the invention relate to a biodegradable stent made of a biological material selected from a group consisting of chitosan, collagen, elastin or tropoelastin, gelatin, fibrin glue, biological sealant, and combination thereof. In a further embodiment, the stent is crosslinked with a crosslinking agent or with ultraviolet irradiation. In another embodiment, the stent is loaded with at least one bioactive agent.

### Example #2

### Low MW Chitosan

As shown in Example #1, chitosan powder is generally with a molecular weight (MW) of about 70,000 or higher (coded as regular or high MW chitosan) and is soluble in acetic acid at about pH 4. In operations, adjust the chitosan solution to approximately pH 5.5 (right before it becomes gelled) with NaOH for shaping, spray coating, or other prototype configuration. However, in a drug-eluting implant, certain bioactive agents, particularly the protein type substrates if added, may not survive the very low pH environment that is required to dissolve high MW chitosan. Therefore, it is one object of the present invention to provide certain type of low MW chitosan that is soluble in acetic acid at a pH higher than about 4, preferably between about 4 and 7, more preferably between about 5 and 7 and most preferably between about 6 and 7. Processes to obtain low MW chitosan and use thereof has been documented (Lin Y H et al., “Preparation of nanoparticles composed of chitosan/poly-γ-glutamic acid and evaluation of their permeability through Caco-2 cells” from National Tsing Hua University, Taiwan) that is incorporated herein by reference. The low MW chitosan has generally an average molecular weight of about 50,000 or lower.

### Example #3

### Chitosan Stent

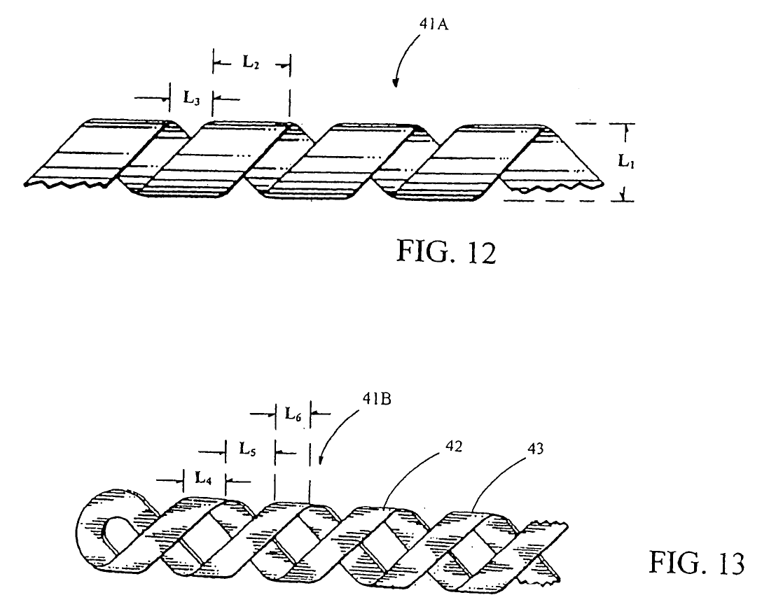

Dissolve chitosan powder in acetic acid at about pH 4 by dispersing 3 grams powder in 50 ml of water containing 0.5 wt % acetic acid. Chitosan (MW: about 70,000) was purchased from Fluka Chemical Co. (Buchs, Switzerland). The chitosan polymer solution was prepared by mechanical stirring at about 600 rpm for about 3 hours until all powder is dissolved. Subsequently, adjust the chitosan solution to approximately pH 5.5 (right before it becomes gelled) with NaOH. Add in at least one bioactive agent of interest into the chitosan solution. While loading the bioactive agent-containing chitosan onto a mold, adjust the environment to pH 7 with NaOH to solidify the chitosan to make a stent. In one example, the mold is a helically bendable hollow mold (such as the one made of silicone or polyurethane-silicone copolymer). During the solidification stage, the mold is promptly bent helically or spirally. After the chitosan is fully solidified, remove the mold to obtain a shaped chitosan pre-product. In another example, a cylindrical mold is used to make a cast chitosan film onto the inner surface of the cylindrical mold. During the solidification stage, the mold is rotated at a desired speed, say, several hundred to several thousand rpm. The cylindrical film, after solidified, is thereafter cut by a spiral knife to make a spiral chitosan pre-product (as shown in FIG. 12). In a third example, the solidifiable solution is made into films, whereas the films are cut into strips of about 2 mm wide. These strips are then wound onto a mandrill and means for forming the helical pre-product is applied, wherein the means may comprise heat set or other change in the environment conditions.

### Example #4

### Chitosan/Glycerol Stent

Dissolve low MW chitosan powder (MW 50,000) in acetic acid by dispersing 3 grams powder in about 50 ml of water containing 0.4 wt % acetic acid and adjust the pH to 5.5 with diluted NaOH. Add 1 ml glycerol to the prepared chitosan solution. Then add at least one bioactive agent of interest into the chitosan solution. While spreading the chitosan-glycerol complex (glycerol-containing chitosan) in a flat dish or beaker, adjust the environment to about pH 7 with NaOH to solidify the chitosan to make a flat sheet or film of appropriate thickness. Then cut the flat sheet or film to from a strip as a pre-shaped chitosan product. In one example, the strip is then wound snugly onto a mandrill and means for forming the helical product is applied, wherein the means may comprise heat set or other change in the environment conditions, such as crosslinking. Additives other than glycerol, such as mineral oils or silicone fluid may be used to make the chitosan composite more flexible than pure chitosan.

In any case, the chitosan pre-product may be further treated with a crosslinking agent, for example genipin, to enhance the biodurability, biocompatibility, but retain certain desired biodegradability. In a preferred embodiment, the chitosan cylindrical film can be cut to make a double spiral or double helix pre-product (as shown in FIG. 13). In another preferred embodiment, after the step of adjusting the chitosan solution to approximately pH 5.5 with NaOH, another substrate or biological material, such as collagen, gelatin, fibrin glue, biological sealant, elastin or tropoelastin, NOCC (N, O, carboxylmethyl chitosan), chitosan-alginate complex, combination thereof, and the like, or phosphorylcholine may be promptly added and well mixed during the manufacturing process. U.S. Pat. No. 5,607,445, the entire contents of which are incorporated herein by reference, discloses a stent having helical and double helical configurations. In one embodiment, the resistance to enzymatic degradation of the biological elastin component in a biodegradable stent can be enhanced by treatment with a crosslinking agent, such as tannic acid (Isenburg J C et al., Biomaterials 2003).

Mi F L, Sung H W and Shyu S S in “Drug release from chitosan-alginate complex beads reinforced by a naturally occurring cross-inking agent” (Carbohydrate Polymers 2002; 48:61-72), the entire contents of which are incorporated herein by reference, discloses drug controlled release characteristics of a chitosan-alginate complex as biological material which is crosslinkable with a crosslinking means for crosslinking the biological material and capable of loaded with at least one drug or bioactive agent.

Fibrin glue is a two-component system of separate solutions of fibrinogen and thrombin/calcium. When the two solutions are combined, the resultant mixture mimics the final stages of the clotting cascade to form a fibrin clot. Fibrin glue is not commercially available in the United States because of the risk of serologically transmitted disease from the preparation of the fibrinogen component. The fibrinogen component can be prepared extemporaneously from autologous, single-donor, or pooled blood. Of course, autologous blood carries essentially no risk of serologically transmitted disease but is also not practical for emergency situations. Fibrin glue is available in Europe under the brand names Beriplast™, Tisseel™, and Tissucol™. Fibrin glue has been used in a wide variety of surgical procedures to repair, seal, and attach tissues in a variety of anatomic sites. The advantage of fibrin glue over other adhesives, such as the cyanoacrylates, is that it is a natural biomaterial that is completely reabsorbed in 2 weeks to 4 weeks. However, the rate of resorption or biodegradation can be slowed down via appropriate crosslinking enabling its use in the drug-eluting stents.

FIG. 12 shows one embodiment of a spiral (helical) biodegradable stent 41A whereas FIG. 13 shows one embodiment of a double helical biodegradable stent 41B according to the principles of the invention. In one embodiment, the spiral stent 41A comprises a spiral film having a cylindrical diameter L₁, a film thickness, a film width L₂ and the spacing L₃ between two helical portions of the film. The film thickness is usually in the range of about 20 microns to 800 microns, preferably 100 to 500 microns. The film width L₂ is usually in the range of about 0.2 mm to 5 mm, preferably 0.5 to 2 mm. The spacing L₃ is usually in the range of about 0.5 to 5 mm, preferably between 0.5 and 2 mm. For non-coronary applications, the upper limit of the aforementioned dimensions could be several times higher. The cylindrical diameter L₁ of the spiral film may expand from a first diameter to a second diameter after the film absorbs liquid or water due to its swelling effect of the biological material used in making the biodegradable stent of the invention. On the other hand, the non-metallic stent made of synthetic polymer, such as non-biodegradable polymer or biodegradable polymer, the diameter change after absorbing liquid (such as water, plasma, or serum) is insignificant. By way of examples, the biodegradable polymer (which is not a biological material) may include, but not limited to, poly(L-lactic acid), polyglycolic acid, poly(D,L-lactide-co-glycolide), poly(ester amides), polycaprolactone, co-polymers thereof, mixtures thereof, and the like. The increase of the cylindrical diameter enhances the retention of the stent against the vessel wall. Some aspects of the invention relate to a biodegradable stent that has a first diameter before contacting water and a second diameter after contacting water, wherein the second diameter is at least 5% more than the first diameter. In one aspect of the invention, it is provided a biodegradable stent that has a first circumference length before contacting water and a second circumference length after contacting water, wherein the second circumference length is at least 5% more than the first circumference length.

In one embodiment, the double helical stent 41B comprises a continuous spiral film that branches to a first spiral film 42 and a second spiral film 43. The first spiral film 42 has a film thickness and a film width L₄ whereas the second spiral film 43 has a film thickness and a film width L₆. The film thickness in either the first spiral film or the second spiral film is usually in the range of about 20 microns to 800 microns, preferably 100 to 500 microns. The film width L₄ or L₆ is usually in the range of about 0.2 mm to 5 mm, preferably 0.5 to 2 mm. The spacing L₅ between the first and second spiral films is usually in the range of about 0.5 to 5 mm, preferably between 0.5 and 2 mm. For non-coronary applications, the upper limit of the aforementioned dimensions could be several times higher.

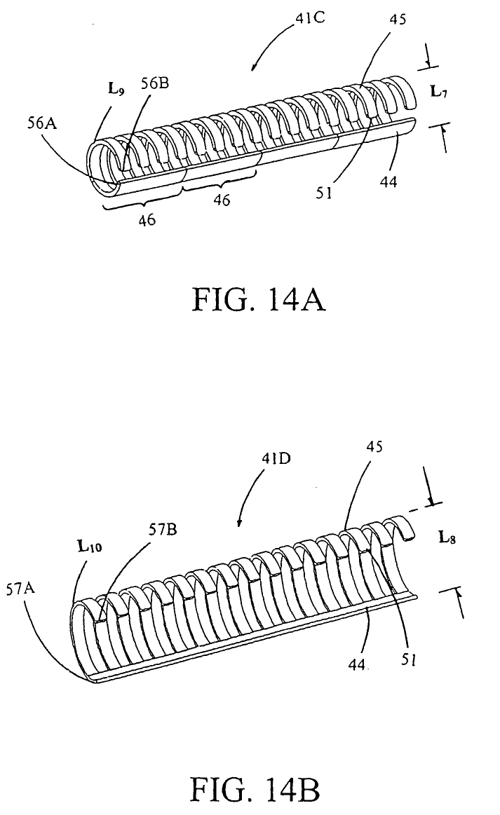

FIGS. 14A, 14B, and 15-18 show one embodiment of an open-ring biodegradable stent. In one aspect, the stent 41C comprises a plurality of open-ring stent members 46. Each stent member 46 comprises a member base 44 and a plurality of ring elements 45, each ring element having a first end secured to the ring base and a second open-ring end 51 that is not connected to the ring base. In one embodiment, all the ring elements may extend from one side of the ring base as shown in FIGS. 14A, 14B, and 15-17. In another embodiment, the ring elements may extend from either side of the ring base as shown in FIG. 18. For illustration, the circumference length L₉ of the ring member is measured from the base point 56A to the open-ring end 56B of that particular ring member. In one illustrative example of FIG. 14A, the biodegradable stent 41C has a first diameter L₇ at the pre-deployment stage.

In an alternate embodiment, at least one ring element 45 is made essentially flat or little curved longitudinally at a free-standing pre-deployment stage. In one embodiment of the pre-deployment stage, at least one ring element is slightly curved to the opposite side of the deployed cylinder. In operations, the stent is first loaded within a catheter sheath having all ring elements curved appropriately and constrained within that sheath void. After delivering to the target lesion site, the stent is delivered out of the catheter sheath and the at least one ring element tends to unravel to fix or anchor the stent in place. In still another embodiment, the stent with at least one ring element that was made essentially flat or lesser curved at a free-standing pre-deployment stage is constrained within a degradable tubular film, such as a gelatin film configured to release the stent in short time via dissolving the gelatin in blood fluid. Some aspects of the invention relate to a crosslinked biodegradable stent comprising at least one bioactive agent, wherein the stent is not in a tubular or cylindrical shape before implantation. In a further embodiment, some aspects of the invention relate to a biodegradable stent comprising at least one bioactive agent, wherein the stent is not in a tubular shape before implantation. In a further embodiment, some aspects of the invention relate to a stent comprising at least one bioactive agent, wherein the stent is not in a tubular shape before implantation.

FIG. 14B shows one embodiment of an open-ring biodegradable stent 41D at a post-deployment stage with reference to its counterpart of the pre-deployed stent 41C, wherein the post-deployed stent 41D has a second diameter L₈ and a second circumference length L₉ which is measured from the base point 57A to the open-ring end 57B of that particular ring member. The open-ring element 45 has a tendency of outward unraveling once it absorbs water. Therefore, the second diameter L₈ could be significantly larger than its counterpart, the first diameter L₇. Similarly, the second circumference length L₁₀ could be slightly or significantly longer than its counterpart, the first circumference length L₉.

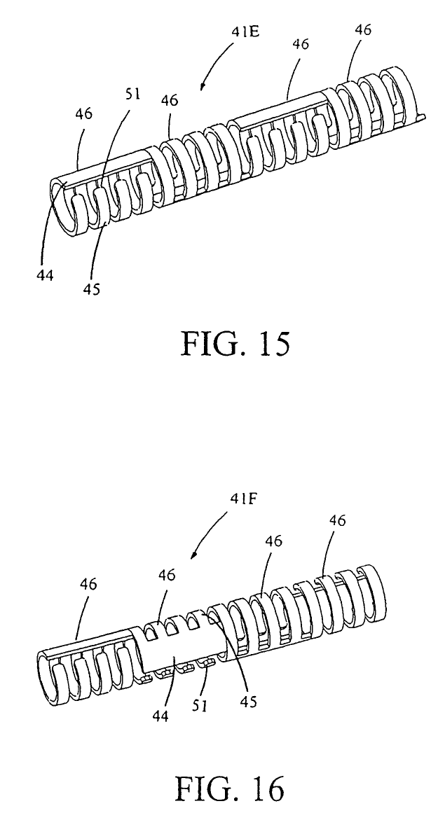

FIGS. 15 and 16 show another embodiments of an open-ring biodegradable stent 41E and/or 41F comprising a plurality of open-ring stent members 46 wherein the bases of the stent members are secured to each other and oriented in a way that the open-ring end 51 of the first stent member 46 may point to a different direction from that of the second stent member. This is to facilitate a more balanced open ring arrangement of an open-ring biodegradable stent. In one embodiment, the stent of the present invention comprises at least one open-ring member 46, at least one open-ring element 45, or at least one ring base 44.

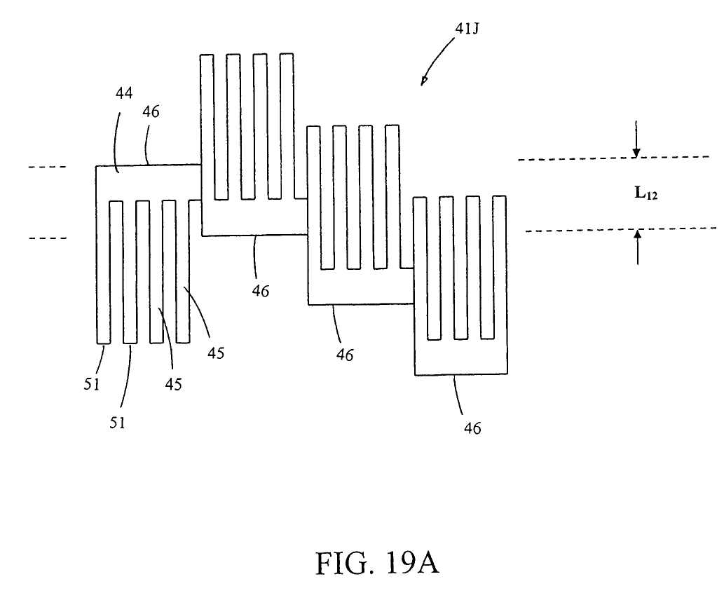

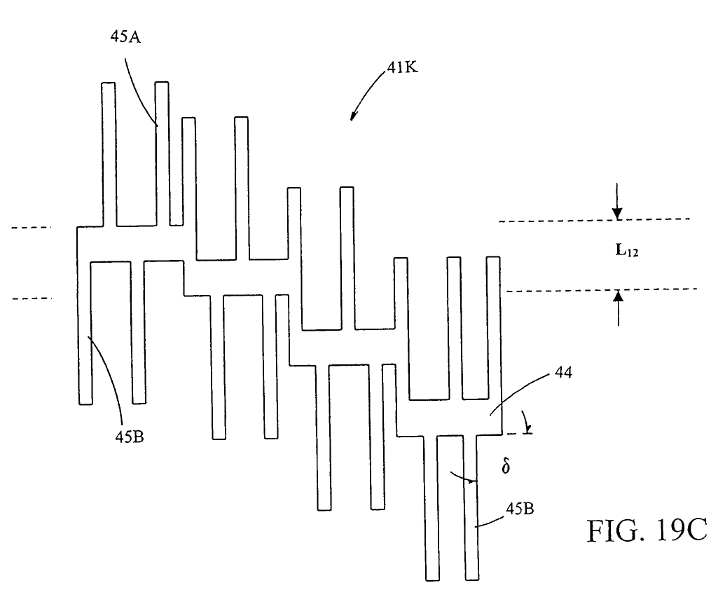

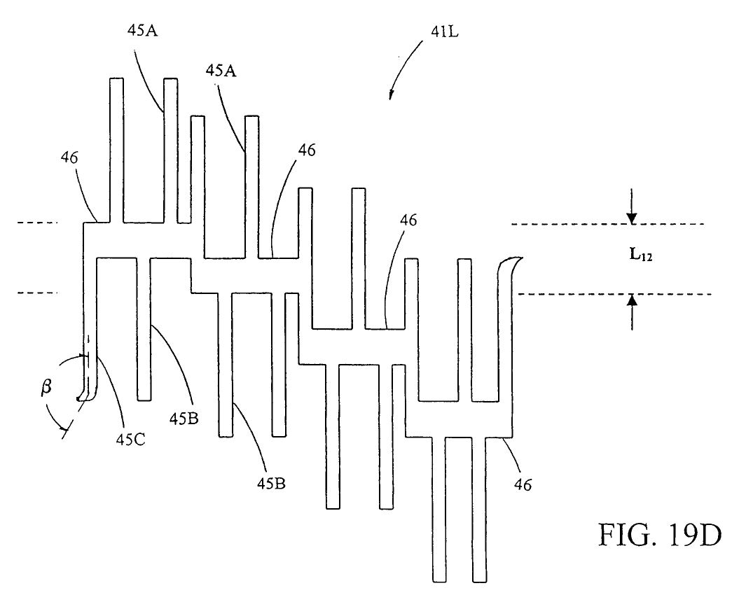

FIG. 19A shows one embodiment of a biodegradable stent not in a tubular shape before implantation according to the principles of the invention. In one embodiment, the stent 41J with all its members 46 is essentially flat. In other words, the stent bases 44, the stent members 46, the open-ring elements 45, and the open-ring ends 51 of a pre-implantation stent 41J are essentially all on the same plane. The stent 41J along with its open-ring elements 45 is to be curved and stored within a catheter sheath 61 or inside a biodegradable tubular holder or film (with a diameter of L₁₂) as shown in FIG. 19B during the stent delivery phase. After deployment out of the catheter sheath, the stent maintains its circular-like shape inside the blood vessel after the open-ring elements 45 unraveled outwardly and contacted the inner wall of the blood vessel.

FIG. 19C shows another embodiment of a stent 41K not in a longitudinally tubular shape before implantation. In one embodiment, the stent 41K is flat so that its stent bases 44, its stent members 46, its open-ring elements 45, and its open-ring ends 51 of the pre-implantation stent are all on the same plane. In a further embodiment, the stent 41K has its open-ring elements spreading to an opposite direction as designated 45A and 45B, wherein at least one open-ring element (for example, 45B) forms an angle (δ) less than 90 degrees with respect to its reference stent base 44. Similarly, the stent 41K along with its open-ring elements 45A, 45B is sized and configured to be loaded and curved within a catheter sheath or inside a biodegradable tubular holder (with a diameter of L₁₂) during the stent delivery phase.

In a further embodiment, most of the stent 41L with its members 46 is flat except for at least one open-ring element. In other words, the stent bases 44, the stent members 46, and most of the open-ring elements 45A, 45B of a pre-implantation stent 41L are on the same plane, except one open-ring element 45C (shown in FIG. 19D) points to a direction at an angle (β) from the reference plane.

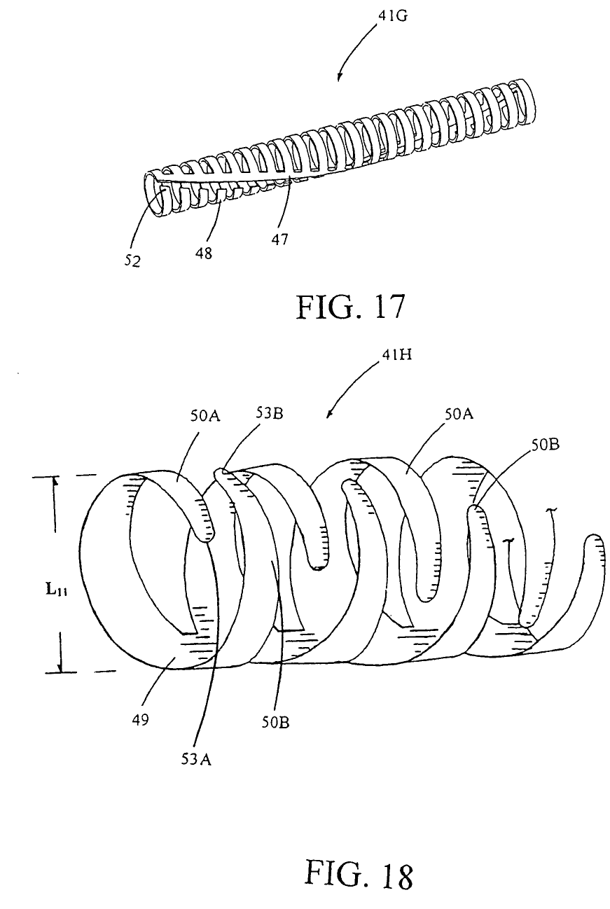

FIG. 17 shows still another embodiment of an open-ring biodegradable stent 41G with spirally or diagonally oriented open pattern according to the principles of the invention. The stent 41G comprises a spirally oriented stent member base 47 and a plurality of ring elements 48, each ring element having a first end secured to the ring base and a second open-ring end 52 that is not connected to the ring base. In one embodiment, the second open-ring ends 52 of the ring elements are configured as a spirally oriented open pattern. In an alternate embodiment, any or all of the open-ring stents of the invention may have the ring elements overlapped the ring base.

FIG. 18 shows one embodiment of an interlocking open-ring biodegradable stent 41H according to the principles of the invention. In one aspect, the interlocking open-ring stent 41H comprises a member base 49 and a plurality of ring elements 50A, 50B, each ring element having a first end secured to the ring base and a second open-ring end 53A, 53B, respectively that is not connected to the ring base 49. In one embodiment, some of the first ring elements 50A may extend from one side of the ring base and some of the second ring elements 50B may extend from an opposite side of the ring base as shown in FIG. 18. In one embodiment, the interlocking open-ring stent 41H comprises an open-ring film element having an equivalent cylindrical diameter L₁₁, a film thickness, a film width and the spacing between any two ring elements. The cylindrical diameter L₁₁ of the ring film element may expand from a first diameter to a second diameter after the film element absorbs liquid or water due to its swelling effect of the crosslinked biological material that is used in fabricating the biodegradable stent of the invention.

### Example #5

### Multiple Layer Stent

Following the steps for making solidifiable chitosan solution or other biological solution loaded with a first bioactive agent in the previous example, the solution is cast to make a chitosan film onto the inner surface of the cylindrical mold. During the solidification stage, the mold is rotated at a desired speed, say, several hundred to several thousand rpm. After the first film is solidified, a second solidifiable chitosan solution or other biological solution loaded with a second bioactive agent can be added on top of the first film and solidified thereafter. By repeating the aforementioned processes, a pre-product with multiple layers of biological material is made. In one embodiment, between each film casting step, the pre-product may be further crosslinked. The cylindrical film with multiple layer and multiple bioactive agents is thereafter cut by a knife in a helical fashion to make a spiral pre-product or a double spiral pre-product. With proper packaging and sterilization, the biodegradable stent is fabricated. Some aspects of the invention relate to a biodegradable stent made of a biological material selected from a group consisting of chitosan, collagen, elastin or tropoelastin, gelatin, fibrin glue, biological sealant, and combination thereof, wherein the stent may comprise a plurality of distinct layers (for example, up to about ten to fifteen layers) made of the biological material, and/or comprise a plurality of layers, each layer is made of the biological material with at least one bioactive agent. In a further aspect of the invention, it provides a method for treating vulnerable plaques of a patient, comprising: providing a biodegradable stent made of a biological material selected from a group consisting of chitosan, collagen, elastin or tropoelastin, gelatin, fibrin glue, biological sealant, and combination thereof; deploying the biodegradable stent to the vulnerable plaques; and releasing the at least one bioactive agent for treating the vulnerable plaques.

### Example #6

Add at least one drug of interest into a collagen solution at 4° C. While loading the drug-containing collagen onto a stent, adjust the environment temperature to about 37° C. to solidify the collagen onto the stent. The process can be accomplished via a continuous assembly line step by providing gradually increasing temperature zones as the device passes by. The loading step can be repeated a few times to increase the thickness or total quantity of the drug-containing collagen. The loading step can be started with a high-dose drug-containing collagen and then loaded with a lower dose drug-containing collagen or vice versa. It is further treated with a crosslinking agent, for example genipin to enhance the biodurability and biocomipatibility. The fixation details could be found elsewhere by Sung et al. (Sung H W, Chang Y, Liang I L, Chang W H and Chen Y C. “Fixation of biological tissues with a naturally occurring crosslinking agent: fixation rate and effects pf pH, temperature, and initial fixative concentration.” J Biomed Mater Res 2000; 52:77-87).

### Example #7

Add drug and stent in a NOCC solution at room temperature. The NOCC (named after “nitrogen Oxygen carboxylmethyl chitosan”) is a chitosan derived compound that is pH sensitive and can be used in drug delivery. This NOCC is water soluble at pH 7. Crosslink the NOCC and drug onto the stent by a crosslinking agent, for example genipin. This is a step of solidification. In one aspect of the present invention, after crosslinking, the drug containing NOCC can be made harder or more solid-like, if needed, by low pH at about 4. The finished stent slowly releases drug when in the body at a pH around neutral.

In a separate study, we evaluated genipin-crosslinked chitosan membranes that were fabricated by means of a casting/solvent evaporation technique (Mi FL et al., J Biomater Sci Polymer Edn 2001; 12(8):835-850). The crosslinked chitosan film which could be used to make a biodegradable spiral stent showed ultimate tensile strength values at about 50-55 MPa. The tensile strength of a dog-bone sample is considered indirectly correlated to the collapse pressure of a cylindrical type stent (Venkatraman S et al., Biomaterials 2003; 24:2105-2111). The ultimate tensile strength of the crosslinked chitosan membranes is about equivalent to that of Venkatraman PLLA4.3 specimen of about 55 MPa (FIG. 4 in Biomaterials 2003; 24:2105-2111) The strain-at-fracture values for the crosslinked chitosan membranes range from about 9 to 22%, which overlaps the strain-at-fracture ranges of 8-12% for Venkatraman PLLA4.3 and PLLA8.4 specimens as shown in FIG. 5 in Biomaterials 2003; 24:2105-2111. Further, the swelling ratio (defined as a ratio of the net weight increase of a swollen material to its corresponding dry weight) for the crosslinked chitosan membranes indicates its desired hydrophilicity as an implant.

### Example #8

Taxol (paclitaxel) is practically water insoluble as some other drugs of interest in this disclosure. Therefore, first mechanically disperse paclitaxel in a collagen solution at about 4° C. Load the drug containing collagen onto a stent and subsequently raise the temperature to about 37° C. to solidify collagen fibers on the stent. The loading step may repeat a plurality of times. Subsequently, crosslink the coated stent with aqueous genipin. The crosslinking on the drug carrier, collagen or chitosan, substantially modify the drug diffusion or eluting rate depending on the degree of crosslinking.

### Example #9

Taxol (paclitaxel) is practically water insoluble as some other drugs of interest in this disclosure. Therefore, first mechanically disperse paclitaxel in a collagen solution at about 4° C. Load the drug containing collagen onto a stent and subsequently raise the temperature to about 37° C. to solidify collagen fibers on the stent. The loading may comprise spray coating, dip coating, plasma coating, painting or other known techniques. The loading step may repeat a plurality of times. The crosslinking on biological material (i.e., the drug carrier, collagen or chitosan,) substantially modify the drug diffusion or eluting rate depending on the degree of crosslinking, wherein the degree of crosslinking of the biological material at a first portion of the stent is different from the degree of crosslinking of the biological material at a second portion or at a third portion of the stent.

### Example #10

Sirolimus is used as a bioactive agent in this example. First mechanically disperse sirolimus in a collagen solution at about 4° C. Load the sirolimus containing collagen onto a stent and subsequently raise the temperature to about 37° C to solidify collagen fibers on the stent. The loading may comprise spray coating, dip coating, plasma coating, painting or other known techniques. The loading step may repeat a plurality of times, wherein each loading step is followed by a crosslinking step, wherein each crosslinking step is either with essentially the same crosslinking degree or with substantially different crosslinking degree. In one alternate embodiment, the degree of crosslinking of collagen at a first portion of the stent is different from the degree of crosslinking of collagen at a second portion of the stent. The resulting sirolimus containing stent with chemically crosslinked collagen is sterilized and packaged for clinical use. By way of example, one preferred sterilization condition may comprise 0.2% peracetic acid and 4% ethanol at room temperature for a period of I minute to a few hours.

Some aspects of the invention provide a medical device, comprising: an apparatus having a surface; a bioactive agent; and biological material loaded onto at least a portion of the surface of the apparatus, the biological material comprising the bioactive agent, wherein the biological material is thereafter crosslinked with a crosslinking agent. The medical device of the invention is further sterilized with a condition comprising a sterilant of peracetic acid about 0.1 to 5% and alcohol (preferably ethanol) about 1 to 20% at a temperature of 5 to 50° C. for a time of about 1 minute to 5 hours.

### Example #11

A collagen solution is used to dip or spray coat a coronary stent to evaluate the effect of the solution surface tension on coating uniformity. A control collagen solution at 10 mg/ml is used to dip coat a stainless steel stent at room temperature. Due to its high surface tension, the collagen tends to cluster or accumulate at the stent corner (where two struts meet) in a thin film. Even after the drying or solidifying step, the collagen at the stent corner is still disproportionately thicker than that at the linear strut portion. In a second experiment, a surfactant (surface tension reducing agent) of 1 μl octanol is added to the control collagen solution. The resulting collagen coated stent shows less cluster at the stent corner than the control run.

The cohesive forces between liquid molecules are responsible for the phenomenon known as surface tension. The molecules at the surface do not have other like molecules on all sides of them and consequently they cohere more strongly to those directly associated with them on the surface. This forms a surface “film” which makes it more difficult to move an object through the surface than to move it when it is completely submersed. Surface tension is typically measured using contact angle techniques in dynes/cm, the force in dynes required to break a film of length 1 cm. Equivalently, it can be stated as surface energy in ergs per square centimeter. Water at 20° C. has a surface tension of 72.8 dynes/cm compared to 22.3 for ethyl alcohol and 465 for mercury. Some aspects of the invention provide a method to load the solidifiable biological material onto at least a portion of a surface of a medical device comprising reducing surface tension of the biological material, wherein the step of loading comprises dip coating, spray coating, co-extrusion, co-molding, plasma coating, or the like.

The “biological substance” made of drug-containing biological material of the present invention and/or the collagen-drug-genipin compound on a stent can be sterilized before use by lyophilization, ethylene oxide sterilization, or sterilized in a series of ethanol solutions, with a gradual increase in concentration from 20% to 75% over a period of several hours. Finally, the drug-loaded stents are rinsed in sterilized saline solution and packaged. The drug carrier, collagen and chitosan, may be fully or partially crosslinked. In one aspect of the present invention, a partially crosslinked collagen/chitosan is biodegradable or bioerodible for drug slow-release.

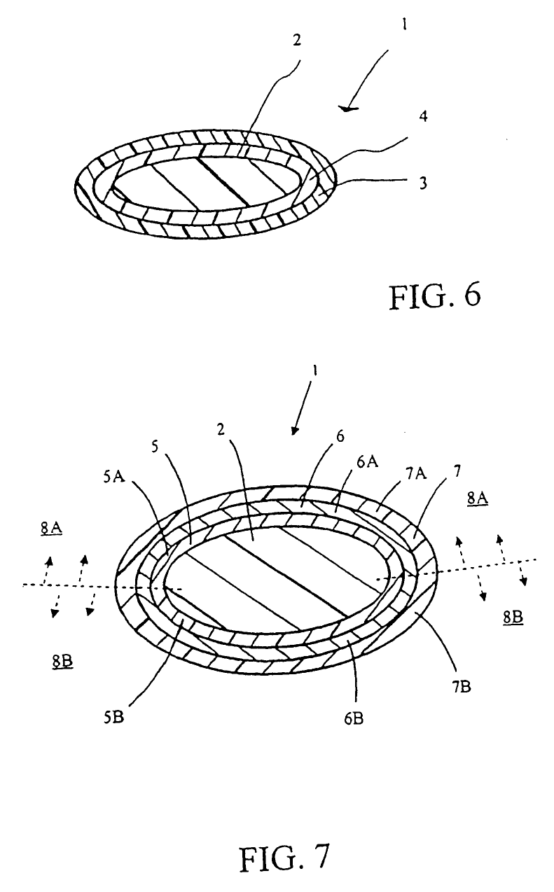

FIG. 6 shows an illustrated example of a cross-sectional view for a medical device of a vascular stent 1 coated with drug-containing collagen 3 crosslinked with genipin according to the principles of the present invention. The stent is generally a mesh type tubular prosthesis made of stainless steel, Nitinol, gold, other metals or plastic material. The vascular stent 1 or a stent strut 2 for non-vascular application may further comprise another layer 4 which is slightly different in composition from the drug-containing collagen layer 3. In some aspect, the layer 4 may have higher drug loading and higher adhesive properties enabling the layer to be securely coated onto the stent strut 2 or the medical device. Due to the barrier properties of the crosslinked collagen, drug could only slowly diffuse out of the crosslinked matrix or released along with biodegraded collagen. This type of drug-eluting stent having collagen carrier chemically treated with genipin is particularly useful in coronary stenting.

Special features for the drug-containing collagen adhesive layer 4 may be characterized by: the layer 4 is securely adhered onto the stent strut; drug is tightly loaded for drug slow release in weeks or months; and collagen is partially crosslinked or fully crosslinked by genipin for stability.

Special features for the drug-containing collagen layer 3 may be characterized by: the layer 3 is securely adhered to layer 4 and vice versa; and drug may be less tightly loaded or collagen may be crosslinked at a lower degree of crosslinkage for drug slow release in days or weeks.

Special features for the drug-loaded collagen and/or drug-loaded chitosan crosslinked by genipin may be characterized by: the crosslinked collagen/chitosan with interpenetrated drug enables drug diffusion at a controlled rate; collagen is tissue-friendly and flexible in deployment; and a crosslinked collagen/chitosan material enhances biocompatibility and controlled biodegradability. The whole process for manufacturing a collagen-drug-genipin or chitosan-drug-genipin compound can be automated in an environmentally controlled facility. Sufficient amount of collagen or drug could be loaded to the exterior side of the stent strut for restenosis mitigation or other therapeutic effects.

FIG. 7 shows one embodiment of a cross-sectional view for a vascular stent 1 with a stent strut 2, wherein the stent surface is coated with a plurality of drug-containing collagen layers 5, 6, 7 that are crosslinked with a crosslinker, or by ultraviolet irradiation or dehydrothermal treatment. FIG. 7 shows the stent outermost surface that is approximately categorized as the tissue contact surface section 8A upon implantation and the blood contact surface section 8B. In one embodiment, the layer thickness of the drug-containing collagen layers 5, 6, 7 in the tissue contact side (that is, 5A, 6A, and 7A) may be different from the layer thickness in the blood contact side (that is, 5B, 6B, and 7B). In another embodiment, there may comprise either none or at least one collagen layer in the blood contact side. Further, the total drug content, drug type, or drug concentration of the drug-containing collagen layers 5, 6, 7 in the tissue contact side (that is, 5A, 6A, and 7A) may be different from the total drug content, drug type, or the drug concentration in the blood contact side (that is, 5B, 6B, and 7B), respectively. In still another embodiment, each of the crosslinking degrees of the drug-containing collagen layers 5, 6, 7 in the tissue contact side (that is, 5A, 6A, and 7A) may be different from the crosslinking degree of the corresponding layer in the blood contact side (that is, 5B, 6B, and 7B).

### Example #12

Paclitaxel is used as a bioactive agent in this example. First step is to prepare a paclitaxel solution (Solution A) by mixing 20 mg paclitaxel in one ml absolute alcohol. The second step is to add Solution A into collagen solution by adjusting to a final pH4 to obtain Solution B, which has a paclitaxel concentration at about 4 mg/ml. Load the paclitaxel containing collagen onto a stent at room temperature and subsequently raise the collagen pH to about 7 to solidify collagen fibers on the stent. The loading may comprise spray coating, dip coating, plasma coating, painting or other known techniques. The loading may comprise a plurality of steps and forms a plurality layers, such as layers 5, 6, 7 in FIG. 7. Each loading step or layer is followed by a crosslinking step, wherein each crosslinking step is either with essentially the same crosslinking degree or with substantially different crosslinking degree. In another embodiment, the total drug content, drug type, or the drug concentration in each loading step may be the same or different from each other depending on the clinical needs. In still another embodiment, the drug amount, drug type, or drug concentration loaded onto each layer may be different depending on the clinical needs. By way of examples, a coronary stent may comprise an outermost layer with anti-thrombogenic agent (for example, heparin, coumadin and the like) to mitigate acute thrombosis concerns, a middle layer with anti-proliferation agent to prevent sub-acute restenosis issues (for example, paclitaxel, everolimus, sirolimus, angiopeptin and the like) or anti-inflammatory agent, and an innermost layer with growth factors or angiogenesis agent to promote chronic endothelialization at the blood vessel lumen. The anti-inflammatory agent may comprise aspirin, lipid lowering statins, fat lowering lipostabil, estrogen and progestin, endothelin receptor antagonist, interleukin-6 antagonist or monoclonal antibodies to VCAM or ICAM.

Lipostabil is phosphatidylcholine, a liquid form of lecithin, an enzyme which occurs naturally in the body. It was first used in the 1950s to dial down climbing cholesterol and triglyceride numbers and is approved for use in Brazil, Germany, Italy and South America. It took Brazilian dermatologist, Patricia Rittes, widely credited with pioneering the treatment often called Lipo-Dissolve, to reincarnate the drug as a pathway to physical perfection. After experimental use as an injectable fat-dissolver by doctors overseas such as Rittes, it started to make its way stateside. Thanks to some anecdotal evidence and off label usage, a few doctors in the United States are now injecting surgery-shy but eager patients in order to send their eye bags packing, whittle pudgy upper arms and reduce other areas often too small to treat with liposuction. A patient gets injected with the drug at the trouble site or sites spaced over the course of several weeks. A topical anesthetic is used at the injection site. Then the patient waits a couple of weeks and goes back in for another round of shots. After the treatments are over and the swelling subsides, one should find a new, fat free area in its wake thanks to the fat dissolving properties of the drug.

Lipostabil is best used for small areas. Some aspects of the invention provide a method for treating a target tissue of vulnerable plaque of a patient, comprising: providing a medical device having a biodegradable apparatus, wherein a biological material loaded onto at least a portion of the surface of the apparatus, the biological material comprising at least one bioactive agent of lipostabil or fat dissolving agent; crosslinking the biological material with a crosslinking agent or with ultraviolet irradiation; and delivering the medical device to the target tissue of vulnerable plaque and releasing the bioactive agent for treating the target tissue. In one embodiment, the degradation rate of the biodegradable apparatus is slower than the degradation rate of the crosslinked biological material. In this case, the therapeutic effects of the bioactive agent goes along with the degradation of the partially crosslinked biological material prior to complete degradation of the biodegradable apparatus. In another embodiment, the degradation rate of the biodegradable apparatus is faster than the degradation rate of the crosslinked biological material. Under the conditions that the partially crosslinked biological material with its entrapped bioactive agent penetrates into the surrounding tissue, the earlier degradation of the biodegradable apparatus makes the lumen surface susceptible for re-endothelialization.

Vulnerable plaque (also known as high-risk plaque, dangerous plaque or unstable plaque) is the atherosclerotic plaque that is vulnerably prone to rupture. The vulnerable plaques also identify all thrombosis-prone plaques and plaques with a high probability of undergoing rapid progression, thus becoming culprit plaques. In most cases, vulnerable plaque is characterized by active inflammation, thin cap with large lipid core, endothelial denudation with superficial platelet aggression, fissured plaque, little vessel narrowing, and other factors. Some aspects of the invention provides a biodegradable stent loaded with at least one bioactive agent having partially crosslinked collagen carrier to treat the vulnerable plaque, wherein the bioactive agent is slow-released in an effective rate over an effective period of time to treat the inflammation or lipid core associated with vulnerable plaque.

### Example #13

Paclitaxel is used as a bioactive agent in this example. Other bioactive agent, such as sirolimus, everolimus, tacrolimus, dexamethasone, ABT-578, paclitaxel, and the like, may substitute for paclitaxel. First step is to prepare a paclitaxel solution (Solution A) by mixing 20 mg paclitaxel in one ml absolute alcohol. The second step is to add 0.15 ml of Solution A and 0.6 ml of 0.5% genipin solution into 4 mg/ml collagen solution by adjusting to a final pH4 to obtain Solution C at a spraying coatable condition, which has a paclitaxel concentration at about 4 mg/ml. Load the paclitaxel containing collagen onto a stent at about 30° C. temperature and subsequently leave the coated stent at 37° C. for a couple of days to solidify, evaporate acetic acid, and crosslink collagen on the stent. The loading may comprise spray coating, dip coating, plasma coating, painting or other known techniques. The loading step may repeat a plurality of times, wherein each loading step is followed by a crosslinking step, and wherein each crosslinking step is either with essentially the same crosslinking degree or with substantially different crosslinking degree. The resulting drug containing stent with chemically crosslinked collagen is sterilized and packaged for clinical use. By way of example, on preferred sterilization condition may comprise 0.2% peracetic acid and 4% ethanol at room temperature for a period of 1 minute to a few hours. Another sterilization method may comprise a conventional ethylene oxide sterilization that is well known to ordinary persons skilled in the art.

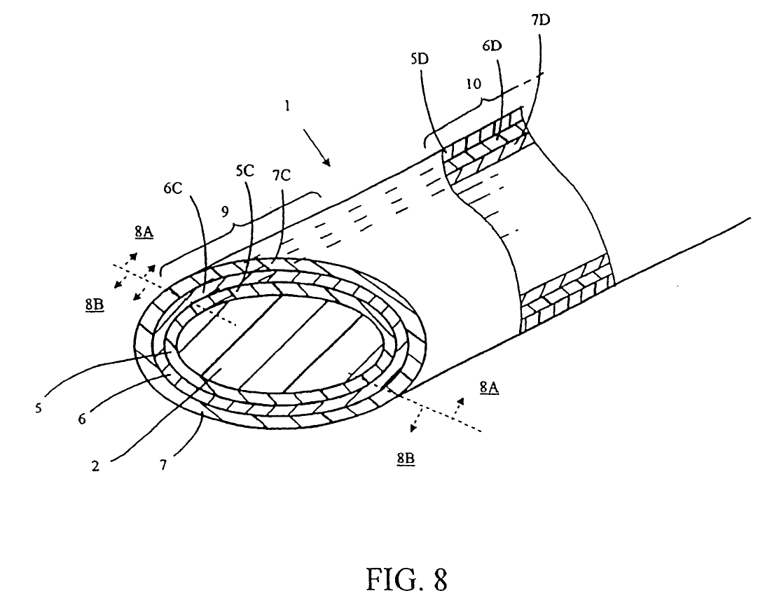

In one alternate embodiment, the crosslinking degree of collagen at a first portion (for example, at a portion 9 adjacent to an end) of the stent is different from the degree of crosslinking of collagen at a second portion (for example, at a second portion 10 spaced away from the end of the first portion 9) of the stent. The stent surface may comprise a first portion, a second portion and other portions, wherein the portion is defined as a surface area of interest, regardless of its size, shape, and location. FIG. 8 shows one embodiment of a longitudinal view for a vascular stent 1 with a stent strut 2, wherein the stent surface is coated with a plurality of drug-containing collagen layers 5, 6, 7 that are crosslinked with a crosslinker, or with ultraviolet irradiation or dehydrothermal treatment. FIG. 8 shows the stent surface or the collagen layer surface that is approximately categorized as the tissue contact surface section 8A upon implantation and the blood contact surface section 8B. In one embodiment, the layer thickness of the drug-containing collagen layers 5, 6, 7 in the first portion 9 (that is, 5C, 6C, and 7C) may be different from the layer thickness in the second portion 10 (that is, 5D, 6D, and 7D). In another embodiment, there may comprise either none or at least one collagen layer in the first portion 9 or the second portion 10. Further, the total drug content, drug type, or drug concentration of the drug-containing collagen layers 5, 6, 7 in the first portion 9 (that is, 5C, 6C, and 7C) may be different from the total drug content, drug type, or drug concentration in the second portion (that is, 5D, 6D, and 7D), respectively. In still another embodiment, each of the crosslinking degree of the drug-containing collagen layers 5, 6, 7 in the first portion (that is, 5C, 6C, and 7C) may be different from the crosslinking degree of the corresponding layer in the second portion (that is, 5D, 6D, and 7D), respectively.

Multi-Layer Drug Loading

Some aspects of the invention provide a drug-eluting implant (for example, a stent) comprising at least one collagen layer (with some or essentially no bioactive agents) that is at least partially crosslinked and at least one drug-containing layer (with some or essentially no collagen or “biological material”). The drug containing layer may contain certain inactive ingredient, such as fillers, diluents, or slow release media, such as biodegradable polymers. The following example illustrates one preferred embodiment for making multi-layer drug-loaded stent. In a further embodiment, different drug may be employed in each drug containing layer.

### Example #14

Sirolimus is used as a bioactive agent in this example. Other bioactive agent, such as everolimus, tacrolimus, dexamethasone, ABT-578, paclitaxel, and the like, may substitute for sirolimus. First, dissolve sirolimus in anhydride ethanol at a concentration about 500 gg/ml (coded as Solution X). Second, prepare collagen solution at a concentration about 5 mg/ml with a pH around 4 that is adjusted by acetic acid (coded as Solution Y). Then load (by spray coating or the like techniques) Solution X onto a rotating stent, followed by another step of loading Solution Y alternately. Each loading step may be separated by appropriate time duration sufficient to maintain certain integrity of the prior layer. In one embodiment, certain degree of mixing or penetrating between layers is desirable. A typical operating condition is for the stent on a horizontal mandrill to rotate at about 144 RPM. After at least one Solution X layer and at least one Solution Y layer are loaded onto a stent, spray a crosslinking solution (coded as Solution Z) by mixing 5% genipin in 70% ethanol for sufficient amount and spraying time, say from a few seconds to several minutes. Thereafter, leave the stent in a moderate temperature (around 37° C.), high humidity environment (close to about 100% relative humidity) for enough time (several minutes to several days) to partially crosslink the collagen portion on the stent. The stent would be ready for use after removing the residuals and sterilization.

Some aspects of the invention provide a drug-eluting stent comprising at least one drug-loaded collagen layer that is at least partially crosslinked. In a further aspect of the invention, the drug-eluting stent comprising at least one drug-loaded collagen layer that is at least partially crosslinked may further comprise at least one drug-containing biodegradable polymer layer. In one embodiment, the collagen layer(s) and the biodegradable polymer layer(s) may overlap each other. In another embodiment, the collagen layer may comprise a minor component of biodegradable polymer whereas the biodegradable polymer layer may comprise a minor component of collagen, wherein the collagen may be partially crosslinked thereafter. The drug in each layer may have different total content, drug concentration, drug type or combination of drug types. As used herein, the term “biodegradable” refers to materials that are bioresorbable and/or degrade and/or break down by mechanical degradation upon interaction with a physiological environment into components that are metabolizable or excretable, over a period of time while maintaining the requisite structural integrity. In one aspect, the biodegradable polymer comprises a biodegradable linkage selected from the group consisting of ester groups, carbonate groups, amide groups, anhydride groups, and orthoester groups. By way of example, poly(ester amides), particularly poly[(8-L-Leu-6)₃-(8-L-Lys(Bz))₁], is well known to one skilled in the art which has been disclosed in U.S. Pat. No. 5,485,496 and elsewhere.

Biodegradable Stent

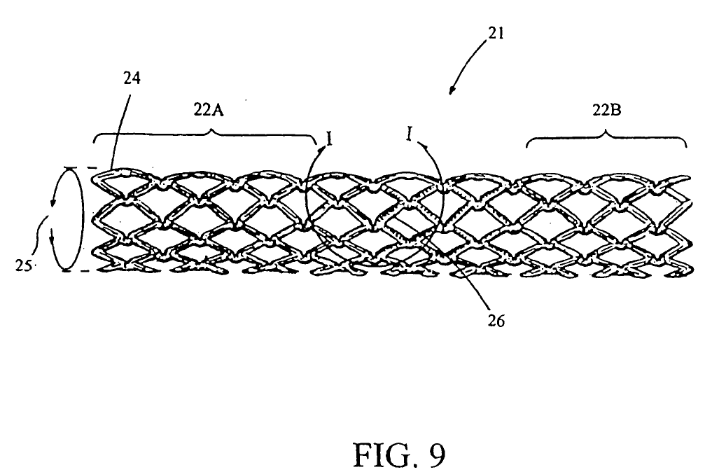

FIG. 9 shows one aspect of a biodegradable stent 21 for treating vulnerable plaques or target tissue of a patient comprising at least two zones, wherein a first supporting zone 22A, 22B comprises at least a portion of continuous circumference (indicated by item 25) of the stent 21, the supporting zone being made of a first biodegradable material 24; and a second therapeutic zone 23 made of a second biodegradable material 26. In another aspect of the invention, the biodegradation rate (BR₂) of the second biodegradable material 26 of the biodegradable stent 21 is equal to or faster than the biodegradation rate (BR₁) of the first biodegradable material 24. In a particular embodiment, the first biodegradable material and/or the second biodegradable material is a shape memory polymer.

U.S. Pat. No. 6,160,084, No. 6,388,043, U.S. Patent Application publication no. 2003/0055198, and no. 2004/0015187, the entire contents of which are incorporated herein by reference, disclose biodegradable shape memory polymer compositions and articles manufactured therefrom. The compositions include at least one hard segment and at least one soft segment. At least one of the hard or soft segments can contain a crosslinkable group, and the segments can be linked by formation of an interpenetrating network or a semi-interpenetrating network, or by physical interactions of the segments. Objects can be formed into a given shape at a temperature above the transition temperature of the hard segment, and cooled to a temperature below the transition temperature of the soft segment. If the object is subsequently formed into a second shape, the object can return to its original shape by heating the object above the transition temperature of the soft segment and below the transition temperature of the hard segment.

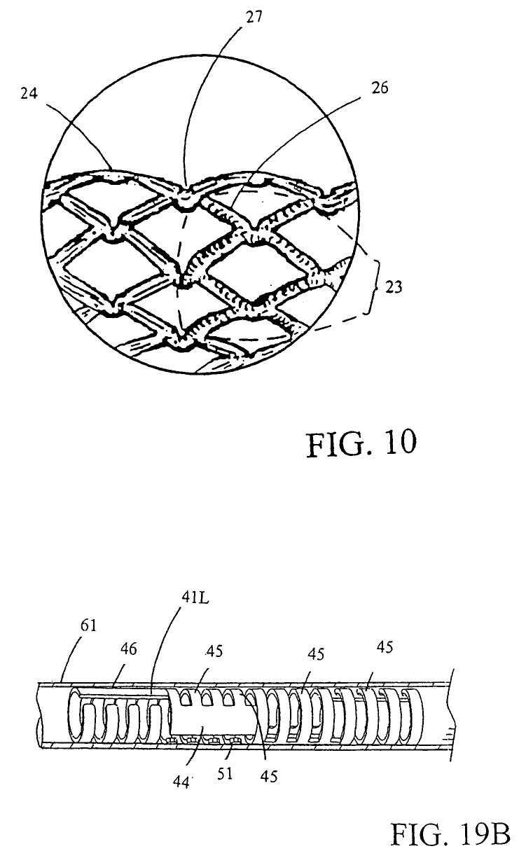

FIG. 10 shows an enlarged view of the biodegradable stent, section I-I of FIG. 9, showing the interface 27 of the first supporting zone 22A and the second therapeutic zone 23. Particularly, the strut of the second biodegradable material 26 meets the strut of the first biodegradable material 24 at the interface 27. In the case that the biodegradation rate for the second biodegradable material (BR₂) is faster than the biodegradation rate of the first biodegradable material (BR₁), the material in the therapeutic zone will biodegrade sooner than the material in the supporting zone. Therefore, during the biodegradation period for the second biodegradable material in the therapeutic zone, the material in the supporting zone still provides appropriate structure integrity for keeping the stent in place. In one aspect, the therapeutic zone may be an isolated island surrounding by the supporting zone. In another aspect, the therapeutic zone can be a part of the continuous circumference of the stent or comprise more than one isolated island.

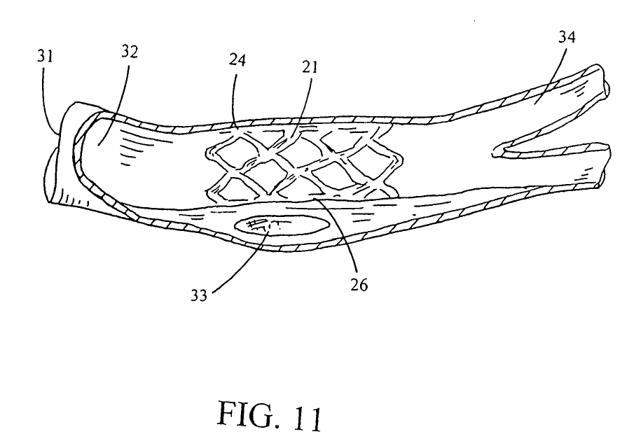

FIG. 11 shows a perspective view of placing the biodegradable stent 21 of the invention at the vulnerable plaque of a patient. The blood vessel 31 of the patient might have some bifurcation 34 and a lipid rich vulnerable plaque 33. Some aspect of the invention provides a method for treating vulnerable plaques of a patient, comprising: (a) providing a biodegradable stent 21 comprising a first supporting zone made of a first biodegradable material 24, wherein the supporting zone comprises at least a portion of continuous circumference of the stent; and a second therapeutic zone made of a second biodegradable material 26, wherein the therapeutic zone comprises at least one bioactive agent; (b) delivering the biodegradable stent to the vulnerable plaques in the lumen 32 of the blood vessel 31; (c) orienting the therapeutic zone at about the luminal surface of the vulnerable plaque 33; and (d) releasing the at least one bioactive agent for treating the vulnerable plaques. In one aspect, the therapeutic zone is capable of covering and treating more than one vulnerable plaque.

Absorbable metal stent was developed by Biotronik (Press release as of Jan. 20, 2005) to treat patients with critical ischemia and lesions in the below-the-knee. The absorbable metal stent is made of an alloy of 90% magnesium (a substance that occurs naturally in the body) and 10% rare earth elements. Clinically, bioabsorption of the stent begins in a week to 10 days and may be fully absorbed within 60 days. Some aspects of the present invention relate to a biodegradable stent loaded with absorbable metal fibers as reinforcing elements sufficient for enhancing the radial strength of the biological stent, wherein at least a portion of the stent comprises a crosslinked biodegradable material. The reinforcing elements would generally constitute about 0.01 to 5% of the base material in weight.

Igaki and Tamai et al. in U.S. Pat. No. 5,733,327, No. 6,045,568, and No. 6,080,177, the entire contents of which are incorporated herein by reference, disclose luminal stents having a holding structure made of knitted yarns of biodegradable polymer fibers that subsequently disappear by being absorbed into the living tissue. Further, Igaki in U.S. Pat. No. 6,200,335 and No. 6,632,242, the entire contents of which are incorporated herein by reference, discloses a stent having a main mid portion and low tenacity portions formed integrally with both ends of the main mid portion. These low tenacity portions are formed so as to have the Young's modulus approximate to that of the vessel of the living body in which is inserted the stent, so that, when the stent is inserted into the vessel, it is possible to prevent stress concentrated portions from being produced in the vessel.

In one aspect, the first biodegradable material or the second biodegradable material of the therapeutic zone of the biodegradable stent of the invention further comprises a biological material, wherein the biological material is crosslinked with a crosslinking agent or with ultraviolet irradiation. In one embodiment, the crosslinking agent is genipin, its analog, derivatives, and combination thereof. In another embodiment, the crosslinking agent is selected from a group consisting of formaldehyde, glutaraldehyde, dialdehyde starch, glyceraldehydes, cyanamide, diimides, diisocyanates, dimethyl adipimidate, carbodiimide, epoxy compound, and mixture thereof. Further, the biological material may be selected from a group consisting of collagen, gelatin, fibrin glue, biological sealant, elastin or tropoelastin, chitosan, N, O, carboxylmethyl chitosan, and mixture thereof, wherein the biological material is a solidifiable substrate, and wherein the biological material may be solidifiable from a phase selected from a group consisting of solution, paste, gel, suspension, colloid, and plasma.

In some aspects, the first biodegradable material or the second biodegradable material of the biodegradable stent is made of a material selected from a group consisting of polylactic acid (PLA), polyglycolic acid (PGA), poly(D,L-lactide-co-glycolide), polycaprolactone, and co-polymers thereof. In another aspect, the first biodegradable material or the second biodegradable material of the biodegradable stent is made of a material selected from a group consisting of polyhydroxy acids, polyalkanoates, polyanhydrides, polyphosphazenes, polyetheresters, polyesteramides, polyesters, and polyorthoesters.

### Example #15

### With ABT-578

In one aspect, the stent as prepared in examples of the invention is made of a metal, such as stainless steel, Nitinol, shape memory metal, cobalt-chromium alloy, other cobalt containing alloy, or the like. On another aspect, the stent as prepared in examples of the invention is made of a non-metallic polymer, such as biodegradable polymer, non-biodegradable polymer, shape memory polymer, or the like. In this example, ABT-578 is used as one of the at least one bioactive agent. In a further embodiment, the ABT-578 containing layer is on the exterior tissue-contacting side, on the interior blood-contacting side, or on the entire surface of the stent. ABT-579 (manufactured by Abbott Laboratories) is a rapamycin analog.

The material in the therapeutic zone of the biodegradable stent may comprise at least one bioactive agent. In one aspect, the at least one bioactive agent is selected from a group consisting of analgesics/antipyretics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, antimicrobials, and anti-infectives. In another aspect, the at least one bioactive agent is selected from a group consisting of actinomycin D, paclitaxel, vincristin, methotrexate, and angiopeptin, batimastat, halofuginone, sirolimus, tacrolimus, everolimus, tranilast, dexamethasone, ABT-578 (manufactured by Abbott Laboratories), and mycophenolic acid. In still another aspect, the at least one bioactive agent is selected from a group consisting of lovastatin, thromboxane A₂ synthetase inhibitors, eicosapentanoic acid, ciprostene, trapidil, angiotensin convening enzyme inhibitors, aspirin, and heparin. In a further aspect, the at least one bioactive agent is selected from a group consisting of allicin, ginseng extract, flavone, ginkgo biloba extract, glycyrrhetinic acid, and proanthocyanides. In some aspect, the at least one bioactive agent comprises ApoA-I Milano or recombinant ApoA-I Milano/phospholipid complexes. In one aspect, the at least one bioactive agent comprises biological cells or endothelial progenitor cells. In some aspects, the at least one bioactive agent comprises lipostabil. In some aspects, the at least one bioactive agent comprises a growth factor, wherein the growth factor is selected from a group consisting of vascular endothelial growth factor, transforming growth factor-beta, insulin-like growth factor, platelet derived growth factor, fibroblast growth factor, and combination thereof.

The polymer stent can be fabricated by extrusion, molding, welding, weaving of fibers. Its manufacturing method may include micromachining or laser machining on a polymer tubing. A preferred method for making a biodegradable stent with at least two zones can be solution molding or thermal molding, which is well known to one skilled in the art, such as exemplified in U.S. Pat. No. 6,200,335.

Suitable biodegradable polymer to be used in the present invention can be found in Handbook of Biodegradable Polymers by Domb et al. (Harwood Academic Publishers: Amsterdam, The Netherlands 1997). Some aspects of the invention provide, in combination, biodegradable and/or bioresorbable polymer as drug carrier and partially crosslinked collagen drug carrier in a drug-eluting stent of the present invention. Some aspects of the invention relate to a medical device, comprising: a biodegradable apparatus having a surface; at least one bioactive agent; and biological material loaded onto at least a portion of the surface of the apparatus, the biological material comprising the at least one bioactive agent, wherein the biological material is crosslinked with a crosslinking agent or with ultraviolet irradiation.

Suitable biodegradable polymer may comprise polylactic acid (PLA), polyglycolic acid (PGA), poly(D,L-lactide-co-glycolide), polycaprolactone, hyaluric acid, adhesive proteins, and co-polymers of these materials as well as composites and combinations thereof and combinations of other biodegradable material. Preferably the materials have been approved by the U.S. Food and Drug Administration. The differentiation of collagen from a biodegradable polymer as a drug carrier is that collagen is crosslinkable after being loaded onto a stent while the polymer is not crosslinkable any more.

One preferred aspect of the invention provides a method for treating a target tissue of a patient comprising: (a) crosslinking a biological material with a crosslinking agent; (b) mixing a bioactive agent with the biological material; (c) loading the biological material onto at least a portion of a surface of a medical device or an apparatus; and (d) delivering the medical device to the target tissue and releasing the bioactive agent for treating the target tissue. In one embodiment, the method comprises a step of solidifying the biological material before the delivering step. In another embodiment, the method further comprises a step of chemically linking the bioactive agent with the biological material through a crosslinker before the solidifying step, wherein the bioactive agent comprises at least a crosslinkable functional group.

In a broader scope of the present invention, the “drug” further comprises bioactive agents or materials which may be used in the present invention include, for example, pharmaceutically active compounds, proteins, oligonucleotides, ribozymes, anti-sense genes, DNA compacting agents, gene/vector systems (i.e., anything that allows for the uptake and expression of nucleic acids), nucleic acids (including, for example, naked DNA, cDNA, RNA, DNA, cDNA, or RNA in a non-infectious vector or in a viral vector which may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic polymers that are selected from a number of types depending on the desired application, including retrovirus, adenovirus, adeno-associated virus, herpes simplex virus, and the like.

For example, biologically active solutes include anti-thrombogenic agents such as heparin, heparin derivatives, urokinase, PPACK (dextrophenylalanine proline arginine chloromethylketone), rapamycin, probucol, and verapamil; angiogenic and anti-angiogenic agents; anti-proliferative agents such as enoxaparin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-mitotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants such as D-Phe-Arg chloromethyl keton, and RGD peptide-containing compound, heparin, antithrombin compounds, platelet receptor antagonists, anti-thrombin antibodies, antiplatelet receptor antibodies, aspirin, prostaglandin inhibitors, platelet inhibitors and tick antiplatelet factors; vascular cell growth promoters such as growth factors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directly against growth factors, bifunctional molecules consisting of a growth factor and a cytotoxin, bifunctional molecules consisting of an antibody and a cytotoxin; cholesterol-lowering agents; vasodilating agents; agents which interfere with endogenous vasoactive mechanisms, and combinations thereof. These and other compounds are applicable to the device and methods of the invention.

U.S. Pat. No. 6,423,682, issued on Jul. 23, 2002 and U.S. Pat. No. 6,485,920, issued on Nov. 26, 2002, the entire contents of both of which are incorporated herein by reference, disclose the compositions of novel human growth factor antagonist proteins and active variants thereof, isolated polynucleotides encoding such polypeptides, including recombinant DNA molecules, cloned genes or degenerate variants thereof, especially naturally occurring variants such as allelic variants, antisense polynucleotide molecules, and antibodies that specifically recognize one or more epitopes present on such polypeptides, as well as hybridomas producing such antibodies function of mitochondria and toxic substances synthesized as a metabolic byproduct within mitochondria of cells. Some aspects of the present invention provide a device comprising solidifiable bioactive agent-containing biological material loaded onto at least a portion of the surface of the device, followed by being crosslinked with a crosslinking agent, wherein the bioactive agent comprises at least one of the above-cited genes.

U.S. Pat. No. 6,476,211, issued on Nov. 5, 2002, the entire contents of which are incorporated herein by reference, discloses human CD39-like protein polynucleotides isolated from cDNA libraries of human fetal liver-spleen and macrophage as well as polypeptides encoded by these polynucleotides and mutants or variants thereof. CD39 (cluster of differentiation 39) is a cell-surface molecule recognized by a “cluster” of monoclonal antibodies that can be used to identify the lineage or stage of differentiation of lymphocytes and thus to distinguish one class of lymphocytes from another. Some aspects of the present invention provide a device comprising solidifiable bioactive agent-containing biological material loaded onto at least a portion of the surface of the device, followed by being crosslinked with a crosslinking agent, wherein the bioactive agent comprises the above-cited human CD39-like protein polynucleotides or the like.

U.S. Pat. No. 5,780,052, issued Jul. 14, 1998, the entire contents of which are incorporated herein by reference, discloses a method of salvaging a target cell from cell death, comprising contacting a target cell having a disrupted cell membrane with a specific affinity reagent-liposome conjugate in an amount effective and for a time sufficient to allow the conjugate to prevent cell death due to membrane disruption. The patent discloses methods of delivering a selected agent into a damaged target cell for diagnosis and therapy, wherein the conjugate comprises a biological agent selected from the group consisting of fibroblastic growth factor-s, angiogenic factors, high energy substrates for the myocardium, antioxidants, cytokines and contrast agents. Some aspects of the present invention provide a device comprising solidifiable bioactive agent-containing biological material loaded onto at least a portion of the surface of the device, followed by being crosslinked with a crosslinking agent, wherein the bioactive agent comprises the above-cited fibroblastic growth factor-β, angiogenic factors, high energy substrates for the myocardium, antioxidants, cytokines and the like.

U.S. Pat. No. 6,475,784, issued on Nov. 5, 2002, the entire contents of which are incorporated herein by reference, discloses a method for polypeptides having anti-angiogenic activity and nucleic acids that encode these polypeptides. The anti-angiogenic polypeptides include at least kringles 1-3 of plasminogen. The patent '784 also provides methods of using the polypeptides and nucleic acids for inhibiting angiogenesis and other conditions characterized by undesirable endothelial cell proliferation. Angiostatin, which is an angiogenesis inhibitor, is a naturally occurring internal cleavage product of plasminogen, wherein human plasminogen has five characteristic protein domains called “kringle structures”. Some aspects of the present invention provide a device comprising solidifiable bioactive agent-containing biological material loaded onto at least a portion of the surface of the device, followed by being crosslinked with a crosslinking agent, wherein the bioactive agent comprises the above-cited anti-angiogenic polypeptides, angiostatin, angiogenesis inhibitor, and the like.

U.S. Pat. No. 6,436,703, issued on Aug. 20, 2002, the entire contents of which are incorporated herein by reference, discloses a method and compositions comprising novel isolated polypeptides, novel isolated polynucleotides encoding such polypeptides, including recombinant DNA molecules, cloned genes or degenerate variants thereof, especially naturally occurring variants such as allelic variants, antisense polynucleotide molecules, and antibodies that specifically recognize one or more epitopes present on such polypeptides, as well as hybridomas producing such antibodies. The compositions in '703 additionally include vectors, including expression vectors, containing the polynucleotides of the invention, cells genetically engineered to contain such polynucleotides and cells genetically engineered to express such polynucleotides. Some aspects of the present invention provide a device comprising solidifiable bioactive agent-containing biological material loaded onto at least a portion of the surface of the device, followed by being crosslinked with a crosslinking agent, wherein the bioactive agent comprises the above-cited antisense polynucleotide molecules and the like.

U.S. Pat. No. 6,451,764, issued on Sep. 17, 2002, the entire contents of which are incorporated herein by reference, discloses a method of treating vascular tissue and promoting angiogenesis in a mammal comprising administering to the mammal an effective amount of the composition comprising VRP (vascular endothelial growth factor-related protein). The disclosure '764 further provides a method for treating trauma affecting the vascular endothelium comprising administering to a mammal suffering from the trauma an effective amount of the composition containing the VRP, or a method for treating a dysfunctional state characterized by lack of activation or lack of inhibition of a receptor for VRP in a mammal. Some aspects of the present invention provide a device comprising solidifiable bioactive agent-containing biological material loaded onto at least a portion of the surface of the device, followed by being crosslinked with a crosslinking agent, wherein the bioactive agent comprises the above-cited inhibitors or receptors for vascular endothelial growth factor-related protein and the like.

It was reported in JAMA. 2003; 290:2292-2300 and 2322-2324, the entire contents of which are incorporated herein by reference, that infusion of Milano Apoprotein causes rapid regression of atherosclerosis in patients with acute coronary syndromes (ACS), according to the results of a preliminary randomized trial published in the November 5 issue of The Journal of the American Medical Association. This intravenous therapy targeting high-density lipoprotein cholesterol (HDL-C) may represent a new approach to the future treatment of atherosclerosis. “Approximately 40 carriers with a naturally occurring variant of apolipoprotein A-I known as ApoA-I Milano are characterized by very low levels of HDL-C, apparent longevity, and much less atherosclerosis than expected for their HDL-C levels,” write Steven E. Nissen, MD, from the Cleveland Clinic Foundation in Ohio, and colleagues. Of 123 patients with ACS, aged 38 to 82 years, who were screened between November 2001 and March 2003 at 10 U.S. centers, 57 patients were randomized. Of 47 patients who completed the protocol, 11 received placebo, 21 received low-dose and 15 received high-dose recombinant ApoA-I Milano/phospholipid complexes (ETC-216) by intravenous infusion at weekly intervals for five doses. Serial intravascular ultrasound measurements within two weeks of ACS and after treatment revealed that the mean percentage of atheroma volume decreased by 1.06% in the combined ETC-216 group compared with an increase of 0.14% in the placebo group. In the combined treatment groups, the absolute reduction in atheroma volume was a 4.2% decrease from baseline.

This initial trial of an exogenously produced HDL mimetic demonstrated significant evidence of rapid regression of atherosclerosis. The authors write, “the potential utility of the new approach must be fully explored in a larger patient population with longer follow-up, assessing a variety of clinical end points, including morbidity and mortality”. In an accompanying editorial, Daniel J. Rader, MD, from the University of Pennsylvania School of Medicine in Philadelphia, discusses several study limitations, including small sample size, short treatment duration, unclear relationship of intravascular ultrasound findings to clinical benefit, and failure to compare infusion of normal ApoA-I with that of ApoA-I Milano.

The mechanisms of action of ApoA-I Milano and phospholipid complex that result in regression of atherosclerosis are unknown but presumably are related to an increase in reverse cholesterol transport from atheromatous lesions to the serum with subsequent modification and removal by the liver (JAMA. 2003; 290:2292-2300). The cysteine substitution for arginine at position 173 for the ApoA-I Milano variant allows dimerization, forming large HDL particles that may be particularly active in reverse cholesterol transport. In vitro experiments have demonstrated increased cholesterol efflux from cholesterol-loaded hepatoma cells incubated with serum from ApoA-I Milano carriers or from transgenic mice. As a result, some day patients with acute coronary syndromes may receive ‘acute induction therapy’ with HDL-based therapies for rapid regression and stabilization of lesions, followed by long-term therapy to prevent the regrowth of these lesions. In this model, long-term HDL-based therapies will still be needed as a vital component of the preventive phase.

The bioactive agent of the present invention further comprises ApoA-I Milano, recombinant ApoA-I Milano/phospholipid complexes (ETC-216), and the like in treating atherosclerosis, both stenotic plaque and vulnerable plaque of a patient for regression and stabilization of lesions. Some aspects of the invention relate to a drug-eluting stent, comprising a biodegradable or non biodegradable stent base coated with at least one layer of partially crosslinked biological material (for example, collagen). In one embodiment, the at least one biological material layer comprises ApoA-I Milano or recombinant ApoA-I Milano/phospholipid complexes. In another embodiment, the at least one biological material layer comprises ApoA-I Milano, recombinant ApoA-I Milano/phospholipid complexes, and other bioactive agent(s). In still another embodiment, a drug-eluting stent of the invention comprises a biodegradable or non biodegradable stent base coated with at least one layer of biodegradable polymer (or combination of biodegradable polymer and partially crosslinked biological material, such as collagen) that is loaded with ApoA-I Milano, or recombinant ApoA-I Milano/phospholipid complexes. In one preferred embodiment, a biodegradable medical device or a biodegradable drug-eluting stent of the invention comprising at least one bioactive agent selected from a group consisting of ApoA-I Milano, recombinant ApoA-I Milano/phospholipid complexes, lipostabil, and combination thereof.

### Example #16

In one aspect, the stent as prepared in examples of the invention is made of a material selected from a group consisting of stainless steel, Nitinol, cobalt-chromium alloy, other cobalt containing alloy, shape memory metal, biodegradable polymer, non-biodegradable polymer, shape memory polymer, or the like. In this example, the stent from either Example 10 or 11 is further coated with PC (phosphorylcholine). In a further embodiment, the PC coating is at least on the inner surface (that is, the blood contacting side after implanted in a blood vessel) of the stent. In another embodiment, the PC coating is at least on the outer surface (that is, the tissue contacting side after implanted in a blood vessel) of the stent. In still another embodiment, the PC coating is over the entire surface of the stent.

PC is found in the inner and outer layers of cell membrane. However, it is the predominant component present in the outer membrane layer, and because it carries both a positive and negative charge (zwitterionic), it is electrically neutral. As a result, the outer layer of the cell membrane does not promote clot formation. When PC is coated on or incorporated on a material, protein and cell adhesion is decreased, clot formation is minimized, inflammatory response is lessened, and fibrous capsule formation is minimized. Some aspects of the invention relate to a drug-eluting stent comprising an immobilized antibody (such as CD34 or the like) that attracts endothelial progenitor cells from the circulating blood stream, resulting in endothelial coverage over and between the stent struts. In a further embodiment, the antibody loading is at least on the inner surface, at least on the outer surface, or over the entire surface of the stent.

Stent Delivery Catheter

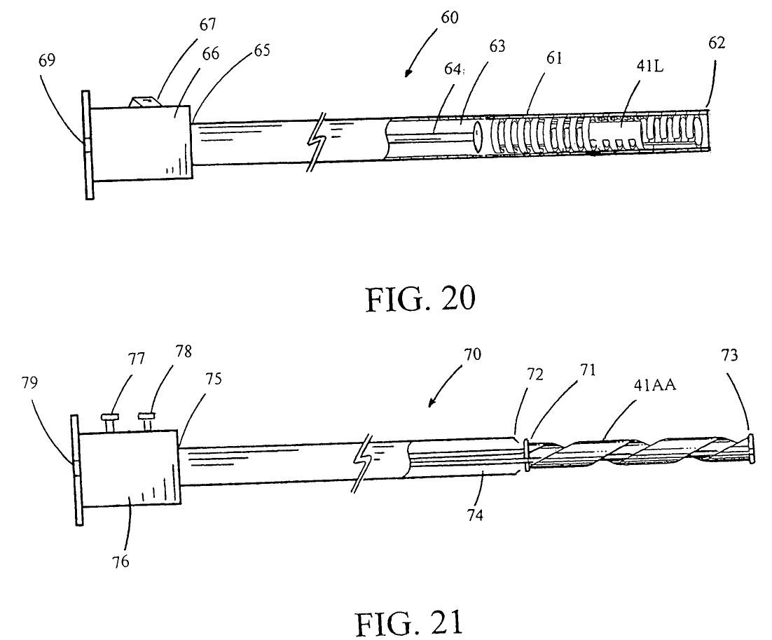

FIGS. 20 and 21 show some embodiments of a delivery apparatus for deploying a biodegradable stent. In one aspect as illustrated in FIG. 20, the delivery catheter 60 comprises a flexible elongate tube with a distal end 62, a proximal end 65, a lumen 64, and a handle 66 attached to the proximal end of the tube. The tube further comprises a distal section with a catheter sheath 61 enclosing a curved biodegradable stent 41L. In one embodiment, a deployment means 67 mounted at the handle for deploying the stent 41L out of the distal end 62 is provided with a plunger-like device 64 which may be a push-pull type device. The handle 66 may further comprise an opening 69 for inserting a guidewire in a standard PTCA operation. A quick exchange type delivery catheter which is well known to one skilled in the art of PTCA catheters is also within the scope of the present invention.

In another aspect as illustrated in FIG. 21, the delivery catheter 70 comprises a flexible elongate tube with a distal end 72, a proximal end 75, a lumen 74, and a handle 76 attached to the proximal end of the tube. The tube further comprises an extended distal section with at least two grasping means 71, 73 for holding each end of the helical stent 41AA in a longitudinal stretched state (with reference to an unstretched helical stent 41A). In one embodiment, each grasping means releasably secures one end of the stent and stretches the stent axially to allow low profile (that is, a smaller stretched diameter than its diameter at a relaxed state) configured and adapted for stent delivery to the lesion site. Thereafter, the stent is released from the grasping means to restore to its original diameter or dimension. In another embodiment, each grasping means releasably secures one end of the stent and stretches the stent by turning one grasping means 73 with reference to the other grasping means 71 to allow low profile (that is, a smaller twisted diameter than its diameter at a relaxed state) configured and adapted for stent delivery to the lesion site. Thereafter, the stent is released from the grasping means to restore to its original diameter or dimension. In a further embodiment, deployment means 77, 78 mounted at the handle for controlling the grasping means 71, 73 to deploy the stent 41AA from the delivery apparatus 70 is provided. The handle 76 may further comprise an opening 79 for inserting a guidewire in a standard PTCA operation.

Certain means for expanding a cardiovascular stent have been disclosed in prior art, such as via a balloon expansion method, a basket expansion method, a self-expanding method, a temperature-sensitive expansion method, a memory-type expansion method, and the like. Those expansion means and their modifications for delivering a biodegradable stent of the present invention are within the scope of the invention. Further, a quick exchange type delivery catheter, an over-the-wire type delivery catheter or their modification are also within the scope of the present invention. The delivery apparatus of the invention is appropriately sized and configured for deploying a biodegradable stent to a lesion site for implantation.

Reversible Crosslinking Agents

In certain medical applications, the carrier is to facilitate drug loading and drug release, particularly in controlled or sustained drug release. In one embodiment, the drug carrier is a biodegradable, biocompatible material, In a further embodiment, the drug carrier is of biological source, not chemically synthesized. In a further embodiment, the drug carrier is crosslinked at a target degree or at a target range of degrees of crosslinking. In a further embodiment, the drug carrier is crosslinked with a crosslinking agent that maintains substantially permanent crosslinking structure. In a further embodiment, the drug carrier is crosslinked with a crosslinking agent that reverses at least a portion of the structure to a non-crosslinking state. A reversible crosslinking agent enables the crosslinked structure to biodegrade earlier than a structure crosslinked with a non-reversible agent configured for certain drug release applications.

It was reported that proanthocyanidin is a natural crosslinking reagent, like genipin, that can crosslink with biological material (J Biomed Mater Res 2003; 65A:118-124). Proanthocyanidin is generally available from grape seeds, nuts, flowers, barks, fruits or vegetables. Proanthocyanidin is part of a specific group of polyphenolic compounds. Four mechanisms for interaction between proanthocyanidin and proteins have been postulated, including covalent interactions, ionic interactions, hydrogen bonding interactions or hydrophobic interactions. The interactions between proanthocyanidin and collagen matrix can be disrupted (reversible) by detergents or hydrogen bond-weakening solvents. It suggests that proanthocyanidin and collagen complex formation involves primarily hydrogen bonding between the protein amide carbonyl and the phenolic hydroxyl (Prog Clin Biol Res 1986; 213:67-76).

The ability of flavanoids and polyphenols to stabilize skin or other tissue against biodegradation leading to tanning (that is, tissue crosslinking) is well documented (J Am Leather Chem Assoc 1944; 39:319). The flavanoids generally include two benzene rings connected by a three carbon chain; may comprise flavonol, isoflanonol, flavone, flavonone, isoflavonone, isoflavone, chalchone and the like. Polyphenolic compounds, such as catechins, from green tea have been shown to reduce inflammation in a murine model of inflammatory arthritis. The types of catechins found in green tea (Camellia sinensis) may include epigallocatechin gallate, epicatechin, epigallocatechin, and epicatechin gallate (J. Nutr. 2002; 132:341-346). The crosslinking capability of catechins with collagen has been demonstrated elsewhere (Experientia 1981; 37:221-223). Other types of polyphenolic compounds may include garlic acid and pentagalloylglucose (tannic acid). Some aspects of the invention relate to a biodegradable stent crosslinked with a reversible crosslinking agent having polyphenolic compounds, such as proanthocyanidin, epigallocatechin gallate, epicatechin, epigallocatechin, and epicatechin gallate.

Fiber-Reinforced Chitosan Biodegradable Stent

Some aspects of the invention relate to a biodegradable vascular stent for treating atherosclerosis comprising a composite material, wherein the composite material comprises a non-metallic base material and a plurality of reinforcing elements for enhancing the radial strength of the vascular stent. In one embodiment, the reinforcing elements are preferably aligned predominantly parallel to each other or aligned in a single plane. In another embodiment, the base material is a crosslinked material, either a biological material or a non-biological polymer. In a further embodiment, the plural reinforcing elements are characterized in that a first Young's modulus of at least a portion of the elements is greater (that is, higher) than a second Young's modulus of the base material. The Young's modulus is generally defined as a ratio of stress to strain for a solid material. In some embodiments, the ratio of reinforcing elements to the base material is in the range of from about 1:102 to 1:1010, preferably in the range of from about 1:10³ to 1:10⁹.

In some embodiments, the reinforcing elements are sized and configured with a longitudinal length and a traverse length, the reinforcing elements being selected from the group consisting of fibers, strips, filaments, elongate meshes, and combination thereof. The traverse length is a diameter of a round fiber or an equivalent diameter (defined as the circumference divided by it) of any non-round shaped element. In an exemplary embodiment, the longitudinal length of the elements is in the range of from about 10 nanometers to 750 μm, preferably in the range of from about 100 nanometers to 500 μm, and most preferably in the range of from about 0.05 μm to 250 μm. In another exemplary embodiment, the traverse length of the elements is in the range of from about 10 nanometers to 750 μm, preferably in the range of from about 100 nanometers to 500 μm, and most preferably in the range of from about 0.1 μm to 500 μm. In another exemplary embodiment, an aspect ratio (longitudinal length/traverse length) of the elements is in the range of from about 2:1 to 100:1. In some further embodiment, the reinforcing elements are made of a biodegradable material selected from the group consisting of metals, metal alloys, metal oxides, polymers, memory polymers, and biological materials. In some embodiment, the reinforcing elements comprise magnesium or magnesium alloy (such as an alloy with magnesium and rare earth elements).

As is known to an ordinary person skilled in the art, reinforcing filaments can be added in heat-resistant ceramics, wherein the reinforcing filament may be selected from the group consisting of iron alloy, aluminum alloy, chromium alloy, cobalt alloy, nickel alloy, and aluminosilicate. It is also known to one skilled in the art that fiber reinforced composite demonstrates high strength or high Young's modulus, wherein the fiber may be selected from the group consisting of carbon fiber, metallic fiber, nylon fiber, aluminosilicate fiber, porous metal fiber, ceramic fiber, and magnesium fiber. U.S. Pat. No. 6,818,288 discloses a fiber-reinforced ceramic composite which comprises at least two layers of a multidirectional woven fiber fabric as reinforcement, with at least 5% of the area of each layer of woven fiber fabric being penetrated by matrix material. U.S. Pat. No. 6,811,861 discloses a reinforced structure having an elongated structural reinforcing strip and a structure to which the strip is affixed by several fasteners inserted through the strip and into the structure, whereas the strip comprises elongated continuous parallel fibers having lengths extending along the length of the strip, nondirectional fibers distributed transversely across the strip; and a polymer matrix affixing and embedding the parallel and nondirectional fibers.

The afore-mentioned metallic or ceramic matrixes can be made into fibers by known fiber-making processes such as the viscous suspension spinning process (Cass, Ceramic Bulletin, Vol. 70, pages 424-429, 1991) with and without metal-containing intercalated graphite, or by the sol-gel process (Klein, Sol-Gel Technology for Thin Films, Fibers, Preforms, Electronics, and Speciality Shapes, Noyes Publications, pages 154-161), and/or by either the slurry or solution extrusion process (Schwartz, Handbook of Structural Ceramics, page 4.55, 1992). The procedures for sample slurry or slip preparation, drawing or extruding material as well as the appropriate drying to remove water, heating to burn off organics, and sintering are discussed in detail in these articles and are well known to one skilled in the art. Metal fibers of the present invention can also be made from other processes, such as micromachining.

U.S. Pat. No. 6,783,712, entire contents of which are incorporated herein by reference, discloses a fiber-reinforced, polymeric implant material useful for tissue engineering, wherein the fibers are preferably aligned predominantly parallel to each other or aligned in a single plane. The implant material comprises a polymeric matrix, preferably a biodegradable matrix, having fibers substantially uniformly distributed therein; U.S. Pat. No. 6,537,312 discloses a biodegradable stent for implantation into a lumen in a human body, wherein the stent is made from a biodegradable fiber, but not a composite material comprising a non-metallic base material and a plurality of reinforcing elements as disclosed in the present invention. U.S. Pat. No. 5,766,710, entire contents of which are incorporated herein by reference, discloses a biodegradable mesh and film stent for use in blood vessels formed of a sheet of a composite mesh material formed of biodegradable high strength polymer fibers bonded together with a second biodegradable adhesive polymer, and laminated on at least one side with a thin film of a third biodegradable polymer. The biodegradable mesh and film material is formed as a sheet and cut in a shape that can be used as a stent, such as a “belt-buckle” type shape, the ends of which can be joined in a contractible, expandable loop.

U.S. Pat. No. 6,585,755, entire contents of which are incorporated herein by reference, discloses an endovascular implant, stent or other medical device formed from a polymeric material, compounded with one or more materials to render the stent visible under both magnetic resonance imaging and x-ray based fluoroscopy procedures. Some aspects of the invention relate to a biodegradable vascular stent for treating atherosclerosis comprising a composite material, wherein the composite material comprises a non-metallic base material and a plurality of reinforcing elements, wherein the stent further comprises one or more imaging materials to render the stent visible under either magnetic resonance imaging or x-ray based fluoroscopy procedure.

Some aspects of the present invention provide a biodegradable vascular stent being configured as a tubular shape, a meshed tubular shape or a non tubular shape prior to being loaded in a delivery apparatus. In a further embodiment, the stent comprises at least one bioactive agent. Some aspects of the present invention relate to a method of treating atherosclerosis of a patient comprising: providing a biodegradable vascular stent comprising a composite material, wherein the composite material comprises a crosslinkable base material and a plurality of reinforcing elements; crosslinking the base material; and delivering the stent to the atherosclerosis for treating the atherosclerosis. In a further embodiment, the reinforcing elements are characterized in that a first Young's modulus of at least a portion of the elements is greater than a second Young's modulus of the base material. In another further embodiment, the vascular stent comprises at least one bioactive agent.

After a first biodegradable (or bioabsorbable) stent is completely degraded or almost completely degraded at the lesion site, a second or more biodegradable stent can be placed at about or adjacent to the same lesion site for subsequent therapy, wherein the later implanted stent may be identical to the prior stent, similar to the prior stent or different from the prior stent with respect to the stent material composition, biodegradation characteristics, stent configuration, and/or loading properties of bioactive agents. This subsequent re-stenting is significant for a biodegradable stent and differentiates from the normal bare metal stent or drug-eluting metal stent with durable metal struts. The re-stenting may be required due to insufficient loading of bioactive agents in the first biodegradable stent, prolonged healing period beyond the drug releasing duration from the first biodegradable stent, a second bioactive agent not compatible with the first biodegradable stent, or a second bioactive agent needed for the second stage of tissue healing. Some aspects of the invention relate to a method for treating vulnerable plaque, atherosclerotic plaque, or a target tissue comprising a first implantation of a biodegradable stent, followed by a second or more implantation at about or adjacent the first implantation site. In one embodiment, the biodegradable stent comprises a biodegradable material selected from a group consisting of biodegradable polymer, biodegradable memory polymer, crosslinked biodegradable biological material, biodegradable metal, and combination thereof. In a future embodiment, the biodegradable stent comprises a non-metallic base material and a plurality of reinforcing elements sufficient for enhancing the radial strength of the vascular stent, wherein the base material is in a continuous phase.

Some aspects of the invention relate to genipin (or its analog, derivatives, and combination thereof) having pharmacological activities in treating hepatic disorders, concentration-dependent inhibition on lipid peroxidation, significant anti-inflammatory effect, and as a specific hydroxyl radical scavenger (Eur J Pharmacology 2004; 495:201-208).

In some embodiment, the method of treating atherosclerosis comprises a step of distal protection by placing a filter apparatus distal to the atherosclerosis for collecting and removing unwanted micro particles. The distal protection is well known to one skilled in the art.

Drug Conjugate

Some aspects of the invention relate to a biodegradable stent comprising a luminal surface portion with a second degree of crosslink, an outer surface portion with a first degree of crosslink, and a wall between the luminal and outer surface portions, wherein the wall comprises a crosslinked material characterized by the first degree of crosslink not less than the second degree of crosslink. In one embodiment, the crosslinked material is a biodegradable material selected from a group consisting of collagen, gelatin, elastin or tropoelastin, chitosan, NOCC, low MW chitosan, fibrin glue, biological sealant, chitosan-alginate complex, chitosan-glycerol complex, and combination thereof. In a further embodiment, the crosslinked material is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

Some aspects of the invention relate to a biodegradable stent comprising crosslinked material, wherein the crosslinked material is a biodegradable material selected from a group consisting of polylactic acid (PLA), polyglycolic acid (PGA), poly(D,L-lactide-co-glycolide), polycaprolactone, poly(amides), poly(ester amides), polyhydroxy acids, polyalkanoates, polyanhydrides, polyphosphazenes, polyetheresters, polyesteramides, polyesters, polyorthoesters, co-polymers thereof, and mixture thereof.

In a further embodiment, the stent further comprises at least one bioactive agent. In one preferred embodiment, the bioactive agent is conjugated to a targeting moiety, wherein the targeting moiety is porphyrin, motexafin lutetium, or a non-porphyrin drug facilitator.

Some aspects of the invention relate to a biodegradable stent comprising a luminal surface portion, an outer surface portion, and a wall between the luminal and outer surface portions, wherein the wall comprises at least one bioactive agent conjugated to a targeting moiety. In one embodiment, the wall comprises a crosslinked material characterized by a degree of crosslink gradually increases from the luminal surface portion to the outer surface portion. In another embodiment, the wall comprises a crosslinked material characterized by a first degree of crosslink at the outer surface portion not less than a second degree of crosslink at the luminal surface portion.

Some aspects of the invention relate to a method of treating vascular atherosclerosis comprising: placing a biodegradable stent proximal to the atherosclerosis, wherein the stent comprises at least one bioactive agent; releasing the bioactive agent; and treating the vascular atherosclerosis distal to the stent. In a further embodiment, the method further comprises a step of providing a distal protection by placing a filter apparatus distal to the atherosclerosis for collecting and removing unwanted micro particles. In a further embodiment, the bioactive agent is conjugated to a targeting moiety.

Photodynamic therapy is a unique modality that combines systemic and local approaches to inhibit plaque formation, cause plaque regress, and enable the diagnosis and stabilization of vulnerable plaque. This approach involves the combination of a chemical photosensitizer and visible light at a specific wavelength to selectively illuminate and activate the photosensitizer, thus leading to the production of singlet radical oxygen species, which mediates apoptosis. Examples of photodynamic therapy using a photosensitizer and an activating light have been disclosed in, for example, U.S. Pat. No. 6,777,402 to Nifantiev et al., and U.S. Pat. No. 6,827,926 to Robinson et al. Further, Robinson et al. in U.S. Pat. No. 6,054,449 and No. 6,794,505, entire contents of which are incorporated herein by reference, discloses a broad class of photosensitive compounds having enhanced in vivo target tissue selectivity and versatility in photodynamic therapy. Preferred photosensitizers are characterized by being retained in the diseased tissue with relatively high and extended localization for a longer time than in normal tissue. The interval between administrations is chosen to be of sufficient duration to allow the photosinsitizer content of normal tissues to drop to a basal or negligible level before the next administration and before irradiation.

A previous animal study has demonstrated that motexafin lutetium, a photosensitizer derived from the porphyrin molecule, binds LDL receptors and is transported into microphage-rich plaque. In the same study, a significant decrease in macrophages and a mild decrease in atheroma were observed without damage to the normal vessel wall in the area of photoactivation (Cardiovascular Research 2001; 49:449-455). Subsequent studies have underscored the affinity of porphyrin derivatives for atheromatous plaques in rabbits and miniswine. Hematoporphyrin and its derivatives provide a new aid for detection of malignant disease and atheromatous plaques because they tend to accumulate in malignant/atheromatous tissue with subsequent red fluorescence for optical viewing (J Thoracic and Cardiovasc Surg 1961; 42:623-629).

Without providing light sources, a photosensitizer may or may not function as a therapeutic agent. Instead, the photosensitizer may function as a “drug facilitator” in a drug conjugate, wherein the drug in the drug conjugate comprises certain class of bioactive agents that is conjugatable (that is, linkable physically or biochemically) to the facilitator. The drug facilitator of the invention may not be limited to photosensitizers. The drug conjugate is intended herein to mean a bioactive complex comprising a drug facilitator and at least a bioactive agent, wherein the drug facilitator (as a targeting moiety) is to facilitate and target the linked bioactive agent to the target tissue for intended therapy. Some aspects of the invention relate to a targeting moiety being capable of preferentially coupling to the atherosclerotic site. Herein “coupling” may also means binding, depositing, linking, bonding and the like. Hematoporphyrin derivative was the first of a number of photosensitizers with demonstrable, selective accumulation within atheromatous plaques.

In some embodiments, the drug conjugate does not include (i.e., it is not coupled either covalently or non-covalently to) an antibody, an enzyme, a hormone, a receptor on a cell surface, or the ligand for a receptor on a cell surface. However, in other embodiments, the drug conjugate can include (i.e., it can be coupled either covalently or non-covalently to) an antibody, an enzyme, a hormone, a receptor on a cell surface, or the ligand for a receptor on a cell surface. These and other useful reactions are discussed in, for example, Hermanson, BIOCONJUGATE TECHNIQUES, Academic Press, San Diego, 1996.

The dosage or concentration of the active component required to produce a favorable therapeutic effect should be less than the level at which the active component produces reactive effects and greater than the level at which non-therapeutic results are obtained. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art. Some aspects of the invention relate to a method of treating vascular atherosclerosis comprising: (A) placing a biodegradable stent proximal to the atherosclerosis, wherein the stent comprises at least one bioactive agent; (B) releasing the at least one bioactive agent; and (C) treating the vascular atherosclerosis distal to the stent. In a further embodiment, the bioactive agent in the biodegradable stent is conjugated to a targeting moiety, wherein the targeting moiety is porphyrin, motexafin lutetium, or a non-porphyrin drug facilitator.

U.S. Pat. No. 6,753,160 to Adair, entire contents of which are incorporated herein by reference, discloses a method for diagnosis and treatment of arteriosclerotic lesions wherein the method is characterized by introducing a chemical compound to the patient, the compound being a complex or conjugate of a targeting moiety (for example, porphyrin) portion and a radioactive portion. The radioactive portion within the compound allows tomographic scanning as well as simultaneous radiation treatment once localized. Some aspects of the present invention relate to a drug compound being a complex or conjugate of a targeting moiety portion and a bioactive (for example, plaque-regressing statins) portion. The targeting moiety is characterized by being retained in the diseased tissue with relatively high and extended localization for a longer time than in normal tissue. The bioactive portion within the compound allows controlled release treatment once localized. The local controlled release as disclosed herein appears more effective to treat the target tissue than from the active agent in systemic circulation. In one embodiment, the method of treating vascular atherosclerosis comprises placing a biodegradable stent proximal (that is, upstream) to the atherosclerosis, wherein the stent releases bioactive agents via diffusion or biodegraded stent material, or conjugate to treat the vascular atherosclerosis distal (downstream) to the stent.

U.S. Pat. No. 6,821,289, entire contents of which are incorporated herein by reference, discloses pheophorbide-a or bacteriopheophorbide as a suitable type of photosensitizer. Both photosensitizers can function as a targeting moiety that can attain a high and extended localization in a diseased tissue in comparison with the surrounding healthy tissue. In certain embodiments, photosensitizers useful for the described methods of this invention include, but are not limited to, members of the following classes of compounds: porphyrins, chlorins, bacteriochlorins, purpurins, phthalocyanines, naphthalocyanines, texaphyrines, and non-tetrapyrrole photosensitizers. For example, the photosensitizer may be, but is not limited to, Photofrin, benzoporphyrin derivatives, tin ethyl etiopurpurin (SnET2), sulfonated chloroaluminum phthalocyanines and methylene blue, and any combination of any or all of the above.

Yock at 2003 TCT meeting in Washington, D.C., reported photodynamic modulation of vulnerable plaque with antrin (that is, motexafin lutetium) in animal and clinical studies. Motexafin lutetium is excitable by light at about a wavelength of 740 nm that penetrates tissue and is water soluble with short plasma half life and no significant phototoxicity. Further, it is preferentially absorbed by atheromatous plaque. Animal study data indicate that motexafin lutetium localizes in atheromatous plaque and in intimal hyperplasia, variably reduces plaque in rabbit models of atherosclerosis, reduces intimal hyperplasia in rat models, and consistently depletes macrophages across animal models. The clinical implications are: reducing plaque cellularity would have a positive effect on restenosis; and a preferential uptake by macrophages could mean stabilization of vulnerable plaque.

Since it has been reported that hypercholesterolemia is due to elevated LDL (hyperlipidemia), the lowering of LDL levels by dietary therapy is attempted. There are several drug classes that are commonly used to lower LDL levels, including bile acid sequestrants, nicotinic acid (niacin), and 3-hydroxy-3-methylglutaryl coenzyme A (HMG CoA) reductase inhibitors. The HMG CoA reductase inhibitors have been termed statins or vastatins. Statins are among the most effective agents currently on the market for hypercholesterolemia, and include pravastatin (Pravchol, Bristol Myers Squibb), atorvastatin (Warner Lambert/Pfizer), simvastatin (Zocor, Merck), lovastatin (Mevacor, Merck), and fluvastatin (Lescol). The current practice is to take statins orally. After much dilution through digestion, absorption, and systemic circulation, the ultimate effects on lowering the LDL or treating local atherosclerosis of a patient is sub-optimal.

Inflammatory cells avidly consume glucose and lactate. Carbob-14 labeled lactate or fluorine-18 labeled fluorodeoxyglucose has been used to evaluate a variety of functions of the brain after preferentially coupled to the inflamed blood vessel wall. Modification of biomolecules (or bioactive agents as disclosed herein) with an “affinity ligand” is important as it provides a means of coupling two entities together for a variety of in vitro and in vivo applications. In one embodiment, the preferred affinity ligands used for coupling to the biomolecule must have a high enough binding constant with a second compound to allow the two coupled entities to remain together for a period of time. An example of an affinity ligand pair is a monoclonal antibody and its hapten.

Modification of the biomolecule or bioactive agent by attachment (a conjugation means) of another molecule to a particular reactive functional group (e.g. amine, sulfhydryl, aldehyde, ketone) may preclude attachment of a second molecule to that group. Thus, if attachment of more than one type of molecule to a biomolecule is desired (to impart two functions), the attachment must be made at a second site using currently available reagents. Since in some applications, it is desirable to have both an affinity ligand and an effector agent (e.g. a moiety that binds/bonds with a radionuclide), site-selective conjugation is precluded. By way of illustration, ¹⁸F-labeled fluorodeoxyglucose is transported into the brain by passive diffusion, where it is phosphorylated and retained within brain tissue, resulting in an indication of glucose metabolism (see M. Blau, Seminars in Nuclear Medicine, Vol. XV, No. 4 (October), 1985). This agent emits positrons and is utilized as an energy substrate by macrophages and monocytes, and it has shown enhanced localization in experimental atherosclerosis models.

Still further, conjugating agents include those which bind to tissue factor, lymphocyte surface antigens or secreted compounds, and other secreted proteins that become entrapped (or lined) within and characteristics of vulnerable plaque. For example, monocyte chemoattractant peptide 1 (MCP1) localizes on receptors upregulated by the macrophages in plaque. Other target substance in plaque include lectins whose receptors are upregulated on endothelial cells that overlay the plaque.

Some aspects of the invention relate to a biodegradable stent comprising a luminal surface portion, an outer surface portion, and a wall between the luminal and outer surface portions, wherein the wall comprises at least one bioactive agent conjugated to a targeting moiety, the targeting moiety having the capability of facilitating (via linking, coupling, binding, adsorption, or other available means physically or biochemically) the bioactive agent to the target tissue. In a further embodiment, the wall comprises a crosslinked material characterized by a degree of crosslink gradually increases from the luminal surface portion to the outer surface portion to first release the bioactive agent at about the luminal surface portion. In an alternate embodiment, the wall comprises a crosslinked material characterized by a first degree of crosslink at the outer surface portion not less than a second degree of crosslink at the luminal surface portion. In a further embodiment, the wall comprises a crosslinked material characterized by a first degree of crosslink at the outer surface portion less than a second degree of crosslink at the luminal surface portion. In some aspects of the invention, the targeting moiety is porphyrin, motexafin lutetium or a non-porphyrin drug facilitator.

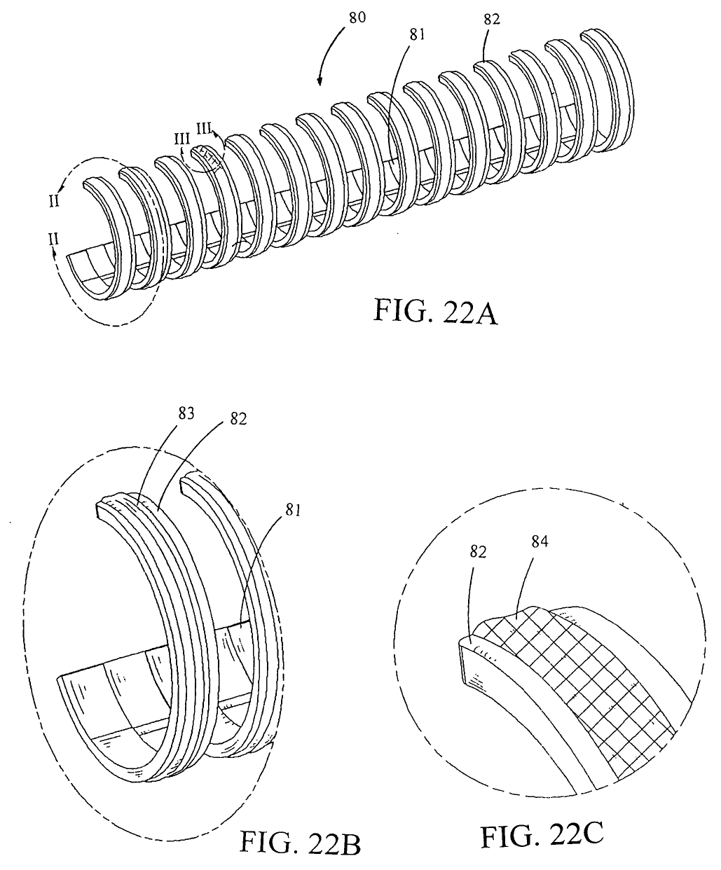

FIG. 22A shows one embodiment of an open-ring biodegradable stent 80 with ring enforcement means for enhancing crushing resistance or hoop strength. FIG. 22B shows a first detailed ring enforcement means, section II-II of FIG. 22A, showing a raised elongate rib 83 along one ring element 82 and FIG. 22C shows a second detailed ring enforcement means, section III-III of FIG. 22A, showing a barrier or a textured surface 84 on the surface of one ring element 82. In some embodiments, the raised rib and textures surface may comprise different material or different properties (for example, physical, mechanical, biochemical, pharmaceutical, therapeutic, and the like) from those of the ring base. Similar to a biodegradable stent 41C, the open-ring biodegradable stent 80 comprises a member base 81 and a plurality of ring elements 82, each ring element having a first end secured to the ring base 81 and a second open-ring end that is not connected to the ring base. In one embodiment, all the ring elements may extend from one side of the ring base as shown in FIG. 22A. In another embodiment, the ring elements may extend from either side of the ring base (not shown).

Some aspects of the invention relate to a biodegradable vascular stent comprising a stent body made of a biodegradable material and a diffusion-restricting barrier material, wherein the barrier material is loaded on at least a portion of the stent body. In a further embodiment, the barrier material is loaded on at least a portion of a surface of the stent body, wherein the barrier material may comprise a non-metallic material for enhancing a radial strength of the stent, wherein the non-metallic material comprises a textured surface on at least a portion of the stent or a raised elongate rib along a circumferential surface of the vascular stent. In a further embodiment, the barrier material is selected from a group consisting of hydrophobic chitosan, poly(L-lactic acid), polyglycolic acid, poly(D,L-lactide-co-glycolide), polycaprolactone, poly(ester amides), mixture thereof, and co-polymers thereof.

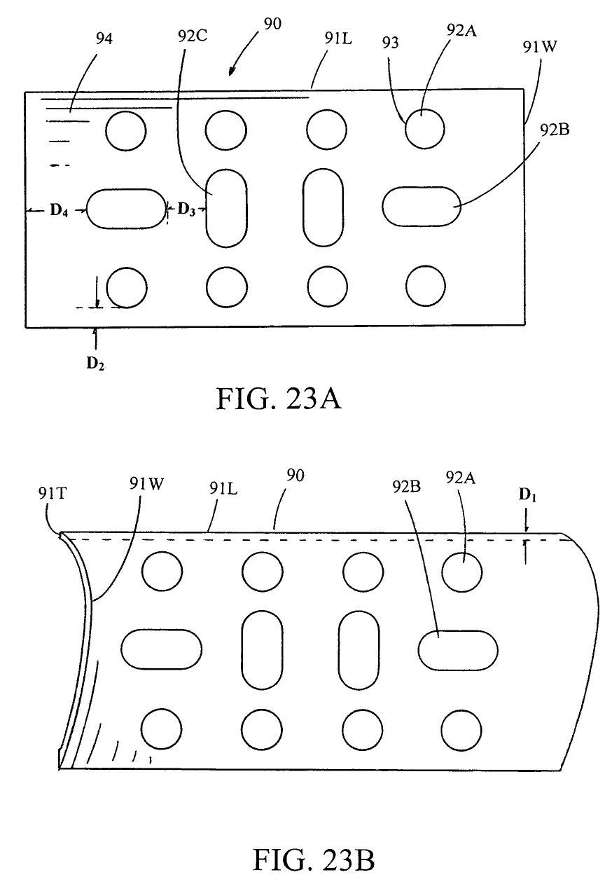

FIG. 23A shows one embodiment of a biodegradable cardiovascular sheet stent 90 in a first position (non-tubular) before implantation or before being inserted into a delivery apparatus, whereas FIG. 23B shows the biodegradable cardiovascular sheet stent of FIG. 23A in a second position being loaded in a delivery apparatus before implantation. In one embodiment, the sheet stent (also known as a “chip” stent“) herein is a quadrilateral polygon. In another embodiment, the sheet stent has four continuous straight peripheral edges, typically in a rectangular, square or parallelogram shape with a uniform depth; at least two opposite edges of the sheet stent may be parallel or not parallel to each other. In some alternate embodiments, the sheet stent may have five or more continuous straight peripheral edges. The sheet stent 90 of the invention (as shown in a second position to be placed in a delivery apparatus or in a next position to be placed in a body conduit) comprises two longitudinal sides 91L, two circumferential (traverse to longitudinal axis) sides 91W, and two radial sides 91T with a thickness D₁. In one embodiment, the sheet stent comprises an inner surface (not shown), an outer surface 94, and a plurality of chip openings 92A, 92B, 92C that are sized, shaped, and configured to allow fluid and nutrient to freely flow between the inner and outer surfaces. After implantation, the chip openings also serve to allow cells to grow therethrough to anchor the sheet stent in place. The peripheral edge 93 of the chip opening 92A is continuously smooth without any cut, acute joint, sudden interface, or sharp point so as to minimize any tear or break therefrom. This unique characteristic differentiates the chip openings from other pores, drilled holes or uncontrolled openings of other devices. The equivalent diameter of the chip openings of the invention may range from about less than 50 microns to a few millimeters (preferably between about 100 to 500 microns).

To avoid blocking any significant arterial branches after implantation, some surface distances (for examples, a distance D₂ between the edge of an opening and a longitudinal side 91L, a distance D₃ between the edges of any two openings, or a distance D₄ between the edge of an opening and a circumferential side 91W) are sized, shaped, and configured to be generally less than about 2 mm, preferably less than about 1 mm, and most preferably less than about 0.5 mm. The sheet stent of the invention may comprise the same composition, formulation, material, additives, bioactive agents, biodegradable properties, and process of manufacture identical to or similar to the biodegradable stents as disclosed in this application.

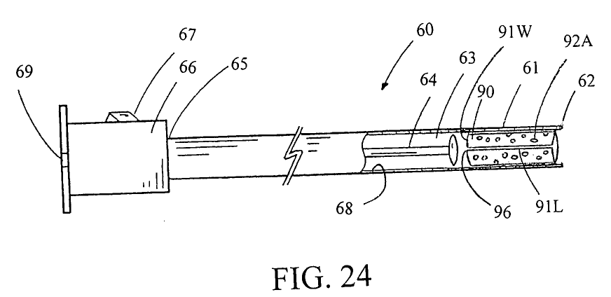

In one aspect as illustrated in FIG. 24, the delivery catheter 60 comprises a flexible elongate tube with a distal end 62, a proximal end 65, a lumen 64, and a handle 66 attached to the proximal end of the tube. The tube further comprises a distal section with a catheter sheath 61 enclosing a curved biodegradable sheet stent 90 for stent delivery, wherein the sheath further comprises an internal surface 68 that may be treated with a friction reducing agent, such as hydrophilic surface coating or Teflon coating. In one embodiment, a deployment means 67 mounted at the handle for deploying the stent 90 out of the distal end 62 is provided with a plunger-like device 64 which may be a push-pull type device for pushing the curved circumferential side 91W forward. In a relative sense, the stent is deployed by holding the plunger against the stent still while pulling the sheath backward. The handle 66 may further comprise a balloon fluid-infusion mechanism or an opening 69 for inserting a guidewire in a standard PTCA operation. A quick exchange type delivery catheter which is well known to one skilled in the art of PTCA catheters is also within the scope of the present invention.

Some aspects of the invention relate to a biodegradable sheet stent 90 comprising an essentially square or rectangular shape in a first pre-implantation position and sized and configured to be a tubular-like (or “curved”, “cylindrical-like”) shape in a second position during deployment in a delivery apparatus or after placement at a target cardiovascular site. The tubular-like shape comprises a small ship clearance or chip slit 96 that is formed between the two edges of the parallel longitudinal sides 91L. The dimension of the chip slit can be predetermined by measuring the target artery size and the dimension of the circumferential side 91W. In one example, a biodegradable sheet stent has the longitudinal dimension of 1.2 cm, a circumferential dimension of 1.1 cm and a sheet thickness of 0.1 cm. This sheet stent is able to be curved as a tubular-like implant and fits into an artery of a 4 mm diameter with a slit dimension of about 1.6 mm. By ways of illustration, a sheet stent (also known as a film stent or chip stent) of the present invention may have a longitudinal length of between about 0.5 to 3.0 cm, a circumferential length of between about 0.5 to 2 cm and a sheet thickness of between about 0.1 to 3 mm. Other dimensions outside of the described ranges are also within the scope of the invention. In one embodiment, the intersection of any two sides (any of longitudinal side, circumferential side, or radial transverse side) may be configured to be a round shape.

While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A biodegradable vascular stent for treating atherosclerosis in a blood vessel comprising a stent body made of a biodegradable material, wherein the stent body is configured to be a polygon shaped sheet before being implanted in the blood vessel.

2. The biodegradable vascular stent of claim 1, said stent body having an internal surface, an external surface, and a plurality of openings between said internal and external surfaces, wherein the stent body is configured to be a non-tubular shape in a first position prior to being loaded in a delivery apparatus for delivering said stent to the blood vessel, and is configured to be a tubular-like shape after being placed in the blood vessel.

3. The biodegradable vascular stent of claim 1, wherein the stent is a quadrilateral polygon.

4. The biodegradable vascular stent of claim 3, wherein the stent is a rectangular or square polygon.

5. The biodegradable vascular stent of claim 1, wherein the biodegradable material is selected from a group consisting of collagen, gelatin, elastin or tropoelastin, chitosan, NOCC, low MW chitosan, fibrin glue, biological sealant, chitosan-alginate complex, chitosan-glycerol complex, and combinations thereof.

6. The biodegradable vascular stent of claim 1, wherein the biodegradable material is further crosslinked with a crosslinking agent or crosslinked with ultraviolet or gamma irradiation.

7. The biodegradable vascular stent of claim 6, wherein the crosslinking agent is selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combinations thereof.

8. The biodegradable vascular stent of claim 1, wherein the biodegradable material is selected from a group consisting of polylactic acid (PLA), polyglycolic acid (PGA), poly(D,L-lactide-co-glycolide), polycaprolactone, poly(amides), poly(ester amides), polyhydroxy acids, polyalkanoates, polyanhydrides, polyphosphazenes, polyetheresters, polyesteramides, polyesters, polyorthoesters, co-polymers thereof, and mixtures thereof.

9. The biodegradable vascular stent of claim 1, wherein the stent body further comprises at least one bioactive agent.

10. The biodegradable vascular stent of claim 9, wherein the bioactive agent is selected from a group consisting of analgesics/antipyretics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, antimicrobials, anti-infectives, and combinations thereof.

11. The biodegradable vascular stent of claim 1, wherein the stent comprises at least one imaging material to render the implant visible under either magnetic resonance imaging or x-ray based fluoroscopy procedure.

12. The biodegradable vascular stent of claim 1, wherein a periphery of the openings is being configured to be continuously smooth without any cut or sharp point.

13. A biodegradable implant comprising a biodegradable material, wherein the implant is sized and configured to be a polygon shape and has an internal surface, an external surface, and a plurality of openings between said internal and external surfaces, the implant body being configured to be an essentially non-tubular shape before being implanted.

14. The biodegradable implant of claim 13, wherein the implant is a rectangular or square polygon.

15. The biodegradable implant of claim 13, wherein the biodegradable material is selected from a group consisting of collagen, gelatin, elastin or tropoelastin, chitosan, NOCC, low MW chitosan, fibrin glue, biological sealant, chitosan-alginate complex, chitosan-glycerol complex, and combinations thereof.

16. The biodegradable implant of claim 13, wherein the biodegradable material is further crosslinked with a crosslinking agent or crosslinked with ultraviolet or gamma irradiation.

17. A biodegradable vascular stent comprising a stent body made of a biodegradable material and a diffusion-restricting barrier material, wherein said barrier material is loaded on at least a portion of the stent body.

18. The biodegradable vascular stent of claim 17, wherein said barrier material is loaded on at least a portion of a surface of the stent body.

19. The biodegradable vascular stent of claim 18, wherein said barrier material comprises a non-metallic material for enhancing a radial strength of the stent, wherein the non-metallic material comprises a textured surface on at least a portion of the stent or a raised elongate rib along a circumferential surface of the vascular stent.

20. The biodegradable vascular stent of claim 17, wherein said barrier material is selected from a group consisting of hydrophobic chitosan, poly(L-lactic acid), polyglycolic acid, poly(D,L-lactide-co-glycolide), polycaprolactone, poly(ester amides), mixtures thereof, and co-polymers thereof.
